(19) United States
(12) Patent Application Publication
(10) Pub. No.: US 2012/0045475 A1
(43) Pub. Date: Feb. 23, 2012

(54) LIVE ATTENUATED VACCINES

(75) Inventors: Glenn Francis Browning, Victoria (AU); Philip Francis Markham, Victoria (AU); Chi-Wen Tseng, Victoria (AU)

(73) Assignee: AUSTRALIAN POULTRY CRC PTY LIMITED, ARMIDALE NEW SOUTH WALES (AU)

(21) Appl. No.: 13/202,534

(22) PCT Filed: Feb. 17, 2010
(86) PCT No.: PCT/AU10/00172

§ 371 Date: Nov. 2, 2011

(30) Foreign Application Priority Data
Feb. 20, 2009 (AU) 2009900736

(51) Int. Cl. A61K 39/02; A61P 37/04; C12N 1/20

(52) U.S. Cl. 424/234.1; 435/252.1; 435/252.5; 435/252.8; 435/252.7; 435/253.3; 435/253.4; 435/253.1; 435/252.3

(57) ABSTRACT

The present invention relates to a bacterium attenuated by a mutation in at least one ABC transporter gene wherein the mutation renders the corresponding ABC transporter protein non-functional and wherein the attenuated bacterium persists in a subject.

### CROSS-REFERENCE TO RELATED APPLICATION

This application claims the benefit of Australian Provisional Patent Application No. 2009900736 filed 20 Feb. 2009 which is incorporated herein by reference in its entirety.

### TECHNICAL FIELD

The present invention relates to the production of live attenuated bacteria which persist in a subject for use in vaccine compositions. In particular the present invention relates to removing the function of at least one ABC-transporter protein of a bacterium to produce an attenuated bacterium which persists in a subject and may be used in a vaccine composition.

### BACKGROUND

Bacterial infections cause significant economic losses in animal industries due to morbidity and mortality and have a significant impact on human health. In particular, respiratory infections and septicaemias are common infectious diseases which may spread throughout a group of animals or humans (subjects) and negatively impact the health of those subjects. This may result in significant productivity losses and ultimately death of the subjects. For example pathogenic bacteria, particularly of the genus Mycoplasma, are a significant cause of respiratory disease. Septicaemia is typically caused by E. coli.

It is known that vaccine compositions comprising attenuated pathogenic micro-organisms such as bacteria or viruses are effective in producing a protective immune response in vaccinated animals and humans. Such live attenuated vaccines are advantageous because after immunization subsequent challenge by the pathogenic organism on which the vaccine is based results in rapid re-stimulation of immune response initially induced by the vaccination. This functions to inhibit the proliferation of the pathogen and prevent the development of clinically relevant disease.

In general, attenuation of pathogenic organisms for use in a vaccine is achieved by complete or partial removal of one or more virulence factors such that the organism is no longer pathogenic. Virulence factors are commonly known as those attributes that directly cause disease and/or allow the organism to persist in the host. Those attributes that promote deleterious host responses are also commonly known to be virulence factors. Typical virulence factors include, for example, toxins, attachment organelles and immune evasion mechanisms. This presents a problem in that many virulence factors are involved in inducing immunity and so deletion of virulence factors compromises the immunogenicity of an organism attenuated in such a way. A live attenuated vaccine preferably remains antigenic and thus capable of inducing a sufficient level of host-immunity while being non-pathogenic. Commonly, live attenuated vaccines do not persist in the subject and this may contribute to reduced immunogenicity of the live attenuated organism on which the vaccine is based.

While some virulence factors, such as toxins, are obvious targets for attenuation, factors not known as virulence factors are not obvious targets for attenuation. The effects of attenuation of those factors on the virulence of an organism is not readily apparent or predictable. Members of the ABC (ATP-binding cassette) transporter superfamily are not commonly thought of as virulence factors. ABC transporters comprise membrane proteins that function to translocate substrates across extra- and intra-cellular membranes. The substances transported by ABC-transporters include peptides, oligopeptides, proteins, metabolic products, lipids, sterols, and drugs. Sequence comparisons of ABC transporters indicate that the genes and proteins of the superfamily are conserved across distantly related phyla.

The present invention is predicated on the surprising finding that a loss of function mutation of an ABC-transporter gene in pathogenic organisms attenuated the pathogenic organisms although the attenuated organisms remained immunogenic and persisted in the subject as assessed in a host-disease model system.

### SUMMARY

In a first aspect there is provided a bacterium attenuated by a mutation in at least one ABC transporter gene wherein the mutation renders the corresponding ABC transporter protein non-functional and wherein the attenuated bacterium persists in a subject.

In a second aspect there is provided a method for attenuating bacteria, the method comprising mutating at least one ABC transporter gene and wherein the attenuated bacteria persists in a subject.

In a third aspect there is provided an immunogenic composition comprising at least one attenuated bacteria of the first aspect.

In a fourth aspect there is provided a vaccine composition comprising an immunogenically effective amount of at least one attenuated bacteria of the first aspect and a pharmacologically acceptable carrier.

In a fifth aspect there is provided a use of vaccine composition for the treatment or prophylaxis of disease wherein the vaccine composition comprises at least one attenuated bacteria of the first aspect.

In a sixth aspect there is provided a method of prevention or amelioration of a disease in a subject, the method comprising administering a therapeutically effective dose of a vaccine composition or immunogenic composition to the subject wherein the vaccine composition or immunogenic composition comprises at least one attenuated bacteria of the first aspect.

In a seventh aspect there is provided a method of prophylaxis of a disease, the method comprising administering a therapeutically effective dose of a vaccine composition or immunogenic composition to a subject in need of prophylaxis wherein is said vaccine composition or immunogenic composition comprises at least one attenuated bacteria of the first aspect.

In one embodiment the mutation may be generated by insertion, deletion, substitution or any combination thereof. The insertion mutation may be made for example by homologous recombination, transposon mutagenesis and sequence tag mutagenesis.

In one embodiment the ABC transporter gene may be a gene encoding for an ABC-peptide transporter protein. For example CvaB, CylB, SpaB, NisT, EpiT, ComA, PedD, LcnC, McbEF, OppD and DppD, and their homologues and in particular the ABC transporter gene may code for OppD.

In one embodiment the bacterium may be selected from the group comprising Avibacterium, Bacillus, Brucella, Bartonella, Bordetella, Burkholderia, Vibrio, Escherichia, Salmonella, Clostridium, Campylobacter, Chalmydia, Coxiella, Erysipelothrix, Francisella, Listeria, Actinobacillus, Haemophilus, Helicobacter, Aeromonas, Pseudomonas, Streptococcus, Shigella, Yersinia, Mycoplasma, Mycobacterium, Mannheimia, Ornithobacterium, Rickettsia, Ureaplasma and Pasteurella. In particular the attenuated bacteria may be Avibacterium paragallinarum, Bordetella avium, Ornithobacterium rhinotracheale, Salmonella enteritidis, Pasteurella multocida, Mannheimia haemolytica, E. coli, Clostridium perfringens, Mycoplasma agalactiae, Mycoplasma antis, Mycoplasma anseris, Mycoplasma imitans, Mycoplasma alkalescens, Mycoplasma arginini, Ureaplasma parvum, Mycoplasma arthritidis, Mycoplasma bovigenitalium, Mycoplasma bovirhinis, Mycoplasma bovis, Mycoplasma bovoculi, Mycoplasma californicum, Mycoplasma capricolum, Mycoplasma dispar, Mycoplasma felis, Mycoplasma fermentans, Mycoplasma genitalium, Mycoplasma hominis, Mycoplasma hyopneumoniae, Mycoplasma hyorhinis, Mycoplasma hyosynoviae, Mycoplasma iowae, Mycoplasma mycoides subsp mycoides large colony and small colony, Mycoplasma gallisepticum, Mycoplasma synoviae, Mycoplasma orale, Mycoplasma penetrans, Mycoplasma ovipneumoniae, Mycoplasma pullorum, Mycoplasma alligatorus, Mycoplasma pneumoniae, Mycoplasma maleagridis, Mycoplasma haemofelis, Mycoplasma haemominutum, Mycoplasma haematoparvum.

In one embodiment the bacterium may be avian pathogenic E. coli strain E956, or Mycoplasma gallisepticum strain Ap3AS.

In one embodiment the live attenuated bacterium may express an heterologous antigen. The heterologous antigen may be encoded by a nucleic acid from another pathogenic organism. The nucleic acid encoding the heterologous antigen may be isolated from the genera selected from the group comprising Avibacterium, Bacillus, Brucella, Bartonella, Bordetella, Burkholderia, Vibrio, Escherichia, Salmonella, Clostridium, Campylobacter, Chalmydia, Coxiella, Erysipelothrix, Francisella, Listeria, Actinobacillus, Haemophilus, Helicobacter, Aeromonas, Pseudomonas, Streptococcus, Shigella, Yersinia, Mycoplasma, Mycobacterium, Mannheimia, Ornithobacterium, Rickettsia, Staphylococci, Ureaplasma and Pasteurella. In particular the nucleic acid encoding the heterologous antigen may be derived from Avibacterium paragallinarum, Bordetella avium, Ornithobacterium rhinotracheale, Salmonella enteritidis, Pasteurella multocida, Mannheimia haemolytica, E. coli, Clostridium perfringens, Mycoplasma hyopneumoniae, Mycoplasma gallisepticum or Mycoplasma synoviae, Avibacterium paragallinarum, Bordetella avium, Ornithobacterium rhinotracheale, Mycoplasma agalactiae, Mycoplasma alkalescens, Mycoplasma antis, Mycoplasma anseris, Mycoplasma imitans, Mycoplasma arginini, Ureaplasma parvum, Mycoplasma arthritidis, Mycoplasma bovigenitalium, Mycoplasma bovirhinis, Mycoplasma bovis, Mycoplasma bovoculi, Mycoplasma californicum, Mycoplasma capricolum, Mycoplasma dispar, Mycoplasma felis, Mycoplasma fermentans, Mycoplasma genitalium, Mycoplasma hominis, Mycoplasma hyopneumoniae, Mycoplasma hyorhinis, Mycoplasma hyosynoviae, Mycoplasma iowae, Mycoplasma mycoides subsp mycoides large colony and small colony, Mycoplasma gallisepticum, Mycoplasma synoviae, Mycoplasma orale, Mycoplasma penetrans, Mycoplasma ovipneumoniae, Mycoplasma pullorum, Mycoplasma alligatorus, Mycoplasma pneumoniae, Mycoplasma, Mycoplasma maleagridis, Mycoplasma haemofelis, Mycoplasma haemominutum, Mycoplasma haematoparvum.

The nucleic acid encoding the heterologous antigen may be isolated from the viruses selected from the group comprising Newcastle Disease virus, Infectious Bronchitis virus, Avian Pneumovirus, Fowlpox, Infectious Bursal Disease, Infectious Laryngotracheitis Virus, Avian Influenza, Duck Virus Hepatitis, Duck Plague, Chicken Infectious Anaemia, Marek's Disease Virus.

The subject is a vertebrate animal including humans, bovines, canines, felines, caprines, ovines, porcines, camelids, equines and avians. The bovine subject may be a cow, ox, bison or buffalo. The canine subject may be a dog. The feline subject may be a cat. The caprine subject may be a goat. The ovine subject may be a sheep. The is porcine subject may be a pig. The camelid subject may be a camel, dromedary, llama, alpaca, vicuña or guanaco. The equine subject may be a horse, donkey, zebra or mule. The avian subject may be any commercially or domestically raised avian. In particular the avian may be a chicken (including bantams), turkey, duck, goose, pheasant, quail, partridge, pigeon, guinea-fowl, ostrich, emu or pea-fowl.

The vaccine compositions and immunogenic compositions may further comprise at least one pharmaceutically acceptable carrier or diluent such as water, saline, culture fluid, stabilisers, carbohydrates, proteins, protein containing agents such as bovine serum or skimmed milk and buffers or any combination thereof.

The stabiliser may be SPGA. The carbohydrates include, for example, sorbitol, mannitol, starch, sucrose, glucose, dextran or combinations thereof. Additionally, proteins such as albumin or casein or protein containing agents such as bovine serum or skimmed milk may be useful as pharmaceutically acceptable carrier or diluents.

Buffers for use as pharmaceutically acceptable carriers or diluents include maleate, phosphate, CABS, piperidine, glycine, citrate, malate, formate, succinate, acetate, propionate, piperazine, pyridine, cacodylate, succinate, MES, histidine, bis-tris, phosphate, ethanolamine, ADA, carbonate, ACES, PIPES, imidazole, BIS-TRIS propane, BES, MOPS, HEPES, TES, MOPSO, MOBS, DIPSO, TAPSO, TEA, pyrophosphate, HEPPSO, POPSO, tricine, hydrazine, glycylglycine, TRIS, EPPS, bicine, HEPBS, TAPS, AMPD, TABS, AMPSO, taurine, borate, CHES, glycine, ammonium hydroxide, CAPSO, carbonate, methylamine, piperazine, CAPS, or any combination thereof.

The vaccine compositions and immunogenic compositions may be lyophilized or freeze-dried.

In some embodiments the vaccine compositions or immunogenic composition may further comprise at least one adjuvant. Examples of adjuvants include Freund's complete adjuvant or Freund's incomplete adjuvant, vitamin E, non-ionic block polymers, muramyldipeptides, saponins, mineral oil, vegetable oil, carbopol aluminium hydroxide, aluminium phosphate, aluminium oxide, oil-emulsions (e.g. of Bayol F® or Marcol 52®), saponins or vitamin-E solubilisate or any combination thereof. In some embodiments the vaccine composition may comprise adjuvants particularly useful for mucosal application for example E. coli heat-labile toxin or Cholera toxin.

The vaccine composition or immunogenic composition may be administered intranasally, opthalmically, intradermally, intraperitoneally, intravenously, subcutaneously, orally, by aerosol (spray vaccination), via the cloaca or intramuscularly. In eye-drop and aerosol administration are preferred when the subject is an avian. Aerosol administration is particularly preferred to administer the vaccine composition or immunogenic composition to large numbers of subjects.

The immunogenic composition or vaccine composition may comprise at least about 10³ to about 10⁵ attenuated bacteria, or about 10⁵ to about 10⁷ attenuated bacteria, or about 10⁷ to about 10⁹ attenuated bacteria, or about 10⁹ to about 10¹¹ attenuated bacteria, or about 10¹¹ to about 10¹³ attenuated bacteria, or about 10¹³ to about 10¹⁵ attenuated bacteria, or about 10¹⁵ to about 10¹⁷ attenuated bacteria, or about 10¹⁷ to about 10¹⁹, or at least about 10¹⁹ attenuated bacteria per dose.

### DEFINITIONS

As used in this application, the singular form “a”, “an” and “the” include plural references unless the context clearly dictates otherwise. For example, the term “an ABC transporter” also includes a plurality of ABC transporters.

In the context of this specification, the term “comprising” means “including principally, but not necessarily solely”. Furthermore, variations of the word “comprising”, such as “comprise” and “comprises”, have correspondingly varied meanings.

As used herein the term “immunogenically effective” means that the amount of live attenuated bacteria administered at vaccination or on administration of an immunogenic composition is sufficient to induce in the subject an effective immune response against virulent forms of the bacterium.

As used herein the terms “treating” and “treatment” refer to any and all uses which remedy a condition or symptoms, prevent the establishment of a condition or disease, or otherwise prevent, hinder, retard, or reverse the progression of a condition or disease or other undesirable symptoms in any way whatsoever.

As used herein the terms “persist” and “persistent” refer to the establishment and/or maintenance of an infection or colonisation of at least part of the subject. Persistence includes a state in which an organism survives in tissues, whether replicating or not. Persistent may or may not be associated with clinically relevant disease.

### BRIEF DESCRIPTION OF THE DRAWINGS

FIG. 1 is a schematic design of components and experimental protocol. A. Basic structure of signature-tagged transposon. B. Design for signature-tagged mutagenesis (STM) experiments.

FIG. 2 Detection of specific tags. Broth cultures showing a colour change were screened by PCR using the P2/P4 primer pair to detect individual signature tags. Each sample was electrophoresed in a 2% agarose gel together with a DNA size marker (pUC18 digested with HaeIII). Lane 1, ST mutant carrying Tag 02; Lane 2, ST mutant carrying Tag 03; Lane 3, ST mutant carrying Tag 04; Lane 4, ST mutant carrying Tag 06; Lane 5, ST mutant carrying Tag 07; Lane 6, negative control. Lane 7, positive control plasmid containing a transposon carrying Tag 02 as template.

FIG. 3 Confirmation of STM transformants. Transformants that showed a colour-change were screening by PCR using P2/P4 primer set to confirm the presence of individual signature tag. Each ample were electrophoresed in a 2% agarose gel along with the DNA size standard of pUC18 digested with HaeIII. Lane 1, recombinant Mg containing Tag02; Lane 2, Tag03 transformant; Lane 3, Tag04 clone; Lane 4, Tag06 transformant; Lane 5, transformant carrying Tag07; +, positive control with Tag02 plasmid as template. −, negative control without template added.

FIG. 4 Experimental design of the confirmatory screening.

FIG. 5 Sequence and Southern blot analysis of ST mutants.

A. Electropherograms obtained by direct sequencing. (I). ST mutant 26-2, which carried a single transposon, with readable sequence into the genome. Mixed sequence signals can be seen starting at the junction of the transposon and host strain genome in ST mutants 15-1 (II) and 25-1 (III).

B. Multiple insertions detected by Southern blotting. Lane 1, ST mutant 15-1; Lane 2, ST mutant 15-2; Lane 3, ST mutant 15-3; Lane 4, ST mutant 25-1; Lane 5, ST mutant 25-2; Lane 6, ST mutant 25-3. The DNA size standards were phage λ DNA digested with HindIII.

FIG. 6 Distribution of RSA and air sac lesion scores in birds in the virulence and infectivity study. A. RSA score at 2 weeks after infection. B. Air sac lesion score. Group 1, negative control; Group 2, ST mutant 04-1 infected; Group 3, ST mutant 33-1 infected; Group 4, ST mutant 03-1 infected; Group 5, ST mutant 26-1 infected; Group 6, ST mutant 18-1 infected; Group 7, ST mutant 20-1 infected; Group 8, ST mutant 22-1 infected; Group 9, positive control (wild-type Ap3AS infected).

FIG. 7 Distribution of RSA and air sac lesion scores of birds. A. RSA score at day 28. B. Air sac lesion score. Sum score of 6 different air sacs. Group 1, negative control; Group 2, positive control (wild-type Ap3AS infected); Group 3, vaccination control (ST mutant 26-1 vaccinated); Group 4, vaccinated (ST mutant 26-1) and challenged (wild-type Ap3AS infected).

FIG. 8. Schematic diagram of PCR constructs used for homologous recombination. The PCR products were generated with the oligonucleotide primers as described. The kanamycin gene was amplified for both oppA and dppD gene constructs using the primer pair TX/TY. Construct arms were generated and added by additional PCR products using WS/WU and WY/WT for oppD or by using 60mer primers WE/WF in PCR for dppD.

FIG. 9. Detection of homologous recombination by PCR. The oligonucleotide primers for dppD (Panel A) and oppD (Panel B) genes were used in PCR to amplify the respective regions in E. coli strains using the primer pair WS/WT for oppD and TZ/UA for dppD: E956 (lane 1 of panels A and B), ΔdppD (lane 1, panel A), and ΔoppD (lane 1, panel B).

FIG. 10. Southern blot of E. coli E956 dppD and oppD knockouts. The kanamycin, dppD and oppD probes were radio labelled and used to probe PstI restricted genomic DNA of the E. coli E956, ΔdppD, and ΔoppD, lanes 1, 2, and 3 respectively.

### DETAILED DESCRIPTION

The present invention relates to live attenuated bacteria which persist in a subject and their use in vaccine compositions. The attenuated bacteria lack at least one functional ABC transporter protein as a result of a mutation in the nucleic acid encoding the ABC transporter protein. The ABC transporter protein may be rendered non-functional by any means but this is typically achieved by mutation of a nucleic acid encoding the ABC transporter protein. Typically the mutation is an insertion, deletion or frameshift mutation or any combination thereof. In some embodiments the live attenuated bacteria may also express an heterologous antigen.

Any type of bacteria may be attenuated in accordance with the invention although the attenuation of pathogenic bacteria of vertebrate animals and in particular avian pathogenic bacteria is preferred.

### Bacteria

In one embodiment, the invention relates to live attenuated bacteria but not limited to the genera Actinobacillus, Aeromonas, Avibacterium, Bacillus, Brucella, Bartonella, Bordetella, Burkholderia, Escherichia, Salmonella, Clostridium, Campylobacter, Chalmydia, Coxiella Erysipelothrix, Francisella, Listeria, Actinobacillus, Haemophilus, Helicobacter, Listeria, Aeromonas, Pasteurella, Streptococcus, Shigella, Yersinia, Mycoplasma, Mycobacterium, Ornithobacterium, Mannheimia, Vibrio, Rickettsia, Pseudomonas, Staphylococci, Ureaplasma and Pasteurella for use in a vaccine composition. These bacterial genera comprise a large number of species that are pathogenic to both avians and a variety of different animals, including humans.

In particular the attenuated bacteria may be but not limited to Avibacterium paragallinarum, Bordetella avium, Ornithobacterium rhinotracheale, Salmonella enteritidis, Pasteurella multocida, Mannheimia haemolytica, E. coli, Clostridium perfringens, Mycoplasma hyopneumoniae, Mycoplasma gallisepticum, Mycoplasma synoviae, Mycoplasma agalactiae, Mycoplasma alkalescens, Mycoplasma antis, Mycoplasma anseris, Mycoplasma imitans, Mycoplasma arginini, Ureaplasma parvum, Mycoplasma arthritidis, Mycoplasma bovigenitalium, Mycoplasma bovirhinis, Mycoplasma Bovis, Mycoplasma bovoculi, Mycoplasma californicum, Mycoplasma capricolum, Mycoplasma dispar, Mycoplasma fells, Mycoplasma fermentans, Mycoplasma genitalium, Mycoplasma hominis, Mycoplasma hyopneumoniae, Mycoplasma hyorhinis, Mycoplasma hyosynoviae, Mycoplasma iowae, Mycoplasma mycoides subsp mycoides large colony and small colony, Mycoplasma gallisepticum, Mycoplasma synoviae, Mycoplasma orale, Mycoplasma penetrans, Mycoplasma ovipneumoniae, Mycoplasma pullorum, Mycoplasma alligatorus, Mycoplasma pneumoniae, Mycoplasma, Mycoplasma maleagridis, Mycoplasma haemofelis, Mycoplasma haemominutum, Mycoplasma haematoparvum.

In one embodiment the live attenuated bacterium may be selected from the group comprising E. coli or Mycoplasma gallisepticum.

The E. coli may be avian pathogenic E. coli E956 and the Mycoplasma gallisepticum may be Mycoplasma gallisepticum Ap3AS.

### Persistence in the Subject

The live attenuated bacteria persist in the subject. Persistence of the attenuated bacteria is preferably assessed in a subject-disease model system but may also be is assessed in subjects to which the vaccine composition or immunogenic composition described herein have been administered. Persistence of the attenuated bacteria may be assessed by way of observation of clinical signs and/or symptoms. Alternatively or in addition persistence may be assessed by determining the presence of the attenuated bacterium in a sample taken from the subject. The sample may be a tissue sample (e.g. blood, skin, hair, buccal scraping) or a sample of bodily secretion or excretion for instance mucus, urine, faeces, lacrimal fluid. Samples may be taken while the subject is alive or at autopsy or necropsy. The presence of the attenuated bacteria in the sample may be assessed by culture, molecular methods such as ELISA or PCR or any method known in the art. Furthermore, observation of affected regions of the subject at autopsy or necropsy may also allow determination that the attenuated bacterium persists in the subject.

### ABC Transporters

ATP-binding cassette transporters (ABC transporter) form one of the largest superfamilies of proteins and genes with representatives in all phyla from prokaryotes to humans. ABC transporters are transmembrane proteins that utilise ATP to transport substances function across cellular membranes. These substances include metabolites peptides, oligopeptides, lipids and sterols, and drugs.

In bacteria, ABC-transporters are subdivided into three main groups on the basis of the type of substrate that each translocates. These groups are the protein transporters, peptide transporters, and systems that transport non-protein substrates.

In accordance with the present invention live attenuated bacteria may be produced by mutating at least one ABC transporter gene of any of these classes such that the bacterium lacks at least one functional ABC transporter protein.

Of particular interest are the ABC-peptide transporters which include for example CvaB (E. coli), CylB (Enterococcus faecalis), SpaB (Bacillus subtilis), NisT (Lactococcus lactis 6F3), EpiT (Staphylococcus epidermidis), ComA (Streptococcus pneumoniae), PedD (Pediococcus acidilactici), LcnC (Lactococcus lactis subsp. lactis), McbEF (E. coli) OppD and DppD (E. coli and M. gallisepticum) and their homologues in other species.

### Subjects

It is contemplated that the subject to which the live attenuated bacteria are to be administered may be any vertebrate animal including humans, bovines, canines, felines, caprines, ovines, porcines, camelids, equines and avians. The bovine subject may be a cow, ox, bison or buffalo. The canine subject may be a dog. The feline subject may be a cat. The caprine subject may be a goat. The ovine subject may be a sheep. The porcine subject may be a pig. The camelid subject may be a camel, dromedary, llama, alpaca, vicuña or guanaco. The equine subject may be a horse, donkey, zebra or mule. The avian subject may be any commercially or domestically raised avian. In particular the avian may be a chicken (including bantams), turkey, duck, goose, pheasant, quail, partridge, pigeon, guinea-fowl, ostrich, emu or pea-fowl.

### Mutations

The mutations utilised to create a non-functional ABC transporter protein may be an insertion, deletion or substitution mutations or a combination thereof, provided that the mutation leads to the failure to express a functional ABC transporter protein. ABC transporter proteins may have multiple functions, for example ATP-binding or oligopeptide binding. A non-functional ABC transporter protein includes an ABC transporter protein that is defective in at least one of its functions.

Such a mutation can be an insertion, a deletion, a substitution mutation or a combination thereof on the proviso that the mutation leads to the failure to express a functional ABC transporter protein. In a preferred embodiment the mutation is a knockout mutation where at least a substantial portion of the nucleic acid encoding the ABC transporter protein is deleted. An insertion mutation may be made for example by homologous recombination, transposon mutagenesis or sequence tag mutagenesis.

Live attenuated bacteria for use according to the invention may be obtained in a number of ways. For example live attenuated bacteria may be prepared by treatment of wild-type bacteria with mutagenic agents such as purine or pyrimidine analogues, ultraviolet light, ionizing radiation, DNA intercalating agents, temperature treatment, transposon mutagenesis. These methods do not target mutations to specific genes and thus necessitate screening of the treated bacteria for attenuation by methods known in the art such as by DNA sequence analysis of target genes in combination with pathogenicity studies.

Recombinant DNA technology using known methods such as site directed mutagenesis may be used to introduce a mutation at a predetermined site of a specific gene, for example oppD. Site directed mutagenesis maybe used to introduce a mutation such as an insertion, a deletion, a replacement of one or more nucleotides such that the mutated gene no longer expresses a functional ABC transporter protein. Such mutations may for instance be made by deletion of a number of nucleic acids.

Deletions of as few as a single base, thus creating a frame shift, can render an ABC protein non-functional. In some embodiments larger numbers of bases are deleted. In other embodiments the majority or all of a gene encoding an ABC transporter protein may be deleted. Mutations that introduce a stop-codon or frame-shift are suitable for obtaining a non-functional ABC transporter protein.

Genes encoding ABC transporter proteins comprise not only the coding sequence, but include regulatory sequences for example promoters. Genes also include regions essential for correct mRNA translation for example ribosome binding sites. Accordingly, the live attenuated bacteria may contain mutations not only in the coding regions but also or alternatively in sequences essential for transcription and translation such as promoters and ribosome binding sites.

In contrast to attenuated bacteria created by spontaneous mutations, attenuated bacteria created by mutations such as deleting fragments of the ABC transporter genes or deleting complete ABC transporter genes or insertion of heterologous DNA-fragments or combinations thereof have the advantage that they will not revert to the wild-type pathogenic bacteria. Accordingly, in a preferred embodiment the invention provides live attenuated bacteria in which at least one ABC transporter gene comprises an insertion and/or a deletion.

### Attenuated Bacteria Expressing Heterologous Antigens

In one embodiment the live attenuated bacteria may be used as carriers for heterologous genes which encode antigens of other pathogenic bacteria or viruses. This allows live attenuated bacteria to be used for invoking an immune response to a plurality of diseases.

The gene encoding the ABC transporter protein may be used as an insertion site for heterologous genes. The use of an ABC transporter gene as an insertion is advantageous because the insertion of a sequence encoding an heterologous antigen both inactivates the ABC transporter protein and introduces a sequence encoding one or more heterologous antigens. The construction of such recombinant bacteria can be done routinely, using standard techniques known in the art.

Another embodiment relates to live attenuated recombinant bacteria selected from the genera Avibacterium, Staphylococcus, Escherichia, Brucella, Salmonella, Bordetella, Burkholderia, Vibrio, Haemophilus, Ornithobacterium, Mannheimia, Pasteurella, Clostridium, Campylobacter, Chalmydia, Coxiella, Erysipelothrix, Francisella, Listeria, Actinobacillus, Haemophilus, Helicobacter, Aeromonas, Pseudomonas, Shigella, Yersinia, Mycoplasma, Mycobacterium, Rickettsia, Ureaplasma and Streptococcus that do not produce a functional ABC transporter and in which a heterologous nucleic acid is inserted. The heterologous nucleic acid may encode an antigen selected from another pathogenic micro-organism or virus. The nucleic acid may encode an antigen or antigens from pathogenic organisms selected from the group comprising Avibacterium paragallinarum, Bordetella avium, Ornithobacterium rhinotracheale, Salmonella enteritidis, Pasteurella multocida, Mannheimia haemolytica, E. coli, Clostridium perfringens, Mycoplasma hyopneumoniae, Mycoplasma gallisepticum or Mycoplasma synoviae, Mycoplasma agalactiae, Mycoplasma alkalescens, Mycoplasma antis, Mycoplasma anseris, Mycoplasma imitans, Mycoplasma arginini, Ureaplasma parvum, Mycoplasma arthritidis, Mycoplasma bovigenitalium, Mycoplasma bovirhinis, Mycoplasma Bovis, Mycoplasma bovoculi, Mycoplasma californicum, Mycoplasma capricolum, Mycoplasma dispar, Mycoplasma fells, Mycoplasma fermentans, Mycoplasma genitalium, Mycoplasma hominis, Mycoplasma hyorhinis, Mycoplasma hyosynoviae, Mycoplasma iowae, Mycoplasma mycoides subsp mycoides large colony and small colony, Mycoplasma orale, Mycoplasma penetrans, Mycoplasma ovipneumoniae, Mycoplasma pullorum, Mycoplasma alligatorus, Mycoplasma pneumoniae, Mycoplasma, Mycoplasma maleagridis, Mycoplasma haemofelis, Mycoplasma haemominutum, Mycoplasma haematoparvum or from viruses but not limited to Newcastle Disease virus, Infectious Bronchitis virus, Avian Pneumovirus, Fowlpox, Infectious Bursal Disease, Infectious Laryngotracheitis Virus, Avian Influenza, Duck Virus Hepatitis, Duck Plague, Chicken Infectious Anaemia and Marek's Disease Virus

In another embodiment the ABC transporter gene may be completely or partially replaced by a nucleic acid a encoding a protein that functions to trigger or enhance an immune response for example interleukin or interferon.

### Vaccine Compositions

The live attenuated bacteria are suitable for use in vaccine compositions. The is vaccine composition may comprise an immunogenically effective amount of a live attenuated bacterium capable of persistence in the subject, and a pharmaceutically acceptable carrier or diluent. The vaccine composition or immunogenic compositions described herein may be administered intranasally, opthalmically, intradermally, intraperitoneally, intravenously, subcutaneously, orally, via the cloaca, by aerosol (spray vaccination) or intramuscularly. In particular eye-drop and aerosol administration are preferred when the subject is an avian. Aerosol administration is particularly preferred when administering the vaccine compositions or immunogenic compositions to large numbers of subjects.

In one form the invention provides live attenuated vaccine compositions for prevention or treatment of animals and humans against infection with a bacterium of which the non-attenuated form of the bacterium comprises an ABC transporter gene.

The pharmaceutically acceptable carrier or diluent may be selected from the group comprising water, saline, culture fluid, stabilisers, carbohydrates, proteins, protein containing agents such as bovine serum or skimmed milk and buffers or any combination thereof.

The stabiliser may be SPGA (per liter, SPGA contains 74.62 g sucrose, 0.52 g KH₂PO₄, 1.25 g K₂HPO₄, 0.912 g potassium glutamate and 10 g serum albumin). Carbohydrates useful as pharmaceutically acceptable carrier or diluents are, for example sorbitol, mannitol, starch, sucrose, glucose, dextran or combinations thereof. Additionally, proteins such as albumin or casein or protein containing agents such as bovine serum or skimmed milk may be useful as pharmaceutically acceptable carrier or diluents.

Buffers as pharmaceutically acceptable carrier or diluents phosphate buffer may be selected from the group comprising maleate, phosphate, CABS (4-(Cyclohexylamino)-1-butanesulfonic acid), piperidine, glycine, citrate, glycylglycine, malate, formate, succinate, acetate, propionate, piperazine, pyridine, cacodylate, succinate, MES (2-(N-Morpholino)ethanesulfonic acid hydrate 4-Morpholineethanesulfonic acid), histidine, bis-tris (2,2-Bis(hydroxymethyl)-2,2′,2″-nitrilotriethanol), phosphate, ethanolamine, ADA (N-(2-Acetamido)iminodiacetic acid, N-(Carbamoylmethyl)iminodiacetic acid), carbonate, ACES (N-(2-Acetamido)-2-aminoethanesulfonic acid), PIPES (1,4-piperazinediethanesulfonic acid), imidazole, BIS-TRIS propane (1,3-Bis [tris(hydroxymethyl)methylamino]propane), BES (N,N-Bis(2-hydroxyethyl)-2-aminoethanesulfonic acid), MOPS (3-(N-Morpholino)propanesulfonic acid), HEPES (4-(2-Hydroxyethyl)piperazine-1-ethanesulfonic acid), TES (2-[(2-Hydroxy-1,1-bis(hydroxymethyl)ethyl)amino]ethanesulfonic acid), MOPSO (3-Morpholino-2-hydroxypropanesulfonic acid), MOBS (4-(N-Morpholino)butanesulfonic acid), DIPSO (3-(N,N-Bis [2-hydroxyethyl]amino)-2-hydroxypropane sulfonic acid), TAPSO (2-Hydroxy-3-[tris(hydroxymethyl)methylamino]-1-propanesulfonic acid), TEA (triethanolamine), pyrophosphate, HEPPSO (4-(2-Hydroxyethyl)piperazine-1-(2-hydroxypropansulfonic acid) Hydrate), POPSO (piperazine-1,4-bis(2-hydroxypropanesulfonic acid) dehydrate), tricine, hydrazine, glycylglycine, TRIS (Tris(hydroxymethyl)aminomethane), EPPS (4-(2-Hydroxyethyl)-1-piperazinepropanesulfonic acid), bicine, HEPBS (N-(2-Hydroxyethyl)piperazine-N′-(4-butanesulfonic acid)), TAPS ([(2-Hydroxy-1,1-bis(hydroxymethyl)ethyl)amino]-1-propanesulfonic acid), AMPD (2-amino-2-methyl-1,3-propanediol), TABS (N-tris(Hydroxymethyl)methyl-4-aminobutanesulfonic acid), AMPSO (N-(1,1-Dimethyl-2-hydroxyethyl)-3-amino-2-hydroxypropanesulfonic acid), taurine, borate, CHES (2-(Cyclohexylamino)ethanesulfonic acid), AMP (2-amino-2-methyl-1-propanol), glycine, ammonium hydroxide, CAPSO (3-(Cyclohexylamino)-2-hydroxy-1-propanesulfonic acid), carbonate, methylamine, piperazine, CAPS (3-(Cyclohexylamino)-1-propanesulfonic acid), or any combination thereof.

With the addition of stabilisers the vaccine composition is suitable for lyophilisation or freeze-drying according to methods known in the art. Accordingly in an embodiment the vaccine composition is freeze-dried or lyophilized.

In some embodiments the vaccine composition may comprise at least one compound having adjuvant activity. Examples of adjuvants suitable for use in vaccine compositions may be selected from the group comprising Freund's complete or Freund's incomplete adjuvant, vitamin E, non-ionic block polymers, muramyldipeptides, saponins, mineral oil, vegetable oil, carbopol aluminium hydroxide, aluminium phosphate, aluminium oxide, oil-emulsions (e.g. of Bayol F® or Marcol 52®), saponins or vitamin-E solubilisate or any combination thereof. In some embodiments the vaccine composition may comprise adjuvants particularly useful for mucosal application for example E. coli heat-labile toxin or Cholera toxin.

The vaccine compositions of the present invention may be formulated for use as aerosols (spray vaccines).

The vaccine compositions of the present invention may be formulated for ophthalmic use, for example in the form of an eye-drop. For example the eye-drops may be aqueous eye drops, non-aqueous eye drops, suspension eye drops or emulsified eye drops. Manufacture of the eye drops is carried out by suspending the live attenuated bacteria in an aqueous solvent such as sterilized distilled water, physiological saline solution or the like or in a non-aqueous solvent such as plant oil including cotton seed oil, soybean oil, sesame oil, peanut oil, mineral oil or the like. Isotonizing agents, pH adjusting agents, thickeners, suspending agents, emulsifiers and preservatives may optionally be added.

Isotonizing agents include for example sodium chloride, boric acid, sodium nitrate, potassium nitrate, D-mannitol, glucose. pH adjusting agents include boric acid, anhydrous sodium sulfite, hydrochloric acid, citric acid, sodium citrate, acetic acid, potassium acetate, sodium carbonate, borax, and any of the buffers listed herein. Thickeners may be selected from the group comprising methyl cellulose, hydroxypropylmethyl cellulose, polyvinyl alcohol, sodium chondroitinsulfate, polyvinylpyrrolidone or any combination thereof.

The suspending agent may be selected from the group comprising polysorbate 80, polyoxyethylene hydrogenated castor oil 60, polyoxy castor oil or combinations thereof. Additionally, examples of emulsifiers include egg yolk lecithin and polysorbate 80. Furthermore preservatives such as benzalkonium chloride, benzethonium chloride, chlorobutanol, phenylethyl alcohol, p-hydroxybenzoates or combinations thereof may be used.

### Dosage

The dosage to be administered will typically vary depending on the age, weight and animal to be vaccinated in addition to the mode of administration and type of pathogen against which vaccination is sought.

The vaccine composition may comprise any dose of bacteria, sufficient to evoke an immune response. For example doses may contain at least 10³ to about 10⁵ attenuated bacteria, or about 10⁵ to about 10⁷ attenuated bacteria, or about 10⁷ to about 10⁹ attenuated bacteria, or about 10⁹ to about 10¹¹ attenuated bacteria, or about 10¹¹ to about 10¹³ attenuated bacteria, or about 10¹³ to about 10¹⁵ attenuated bacteria, or about 10¹⁵ to about 10¹⁷ attenuated bacteria, or about 10¹⁷ to about 10¹⁹, or at least about 10¹⁹ attenuated bacteria.

In one embodiment the vaccine composition may be delivered as an aerosol. The dose for treating 1 cubic meter may contain at least 10⁶ to about 10⁸ attenuated bacteria or about 10⁸ to about 10¹⁰ attenuated bacteria, or about 10¹⁰ to about 10¹², or about 10¹² to about 10¹⁴ attenuated bacteria, or about 10¹⁶ to about 10¹⁸ attenuated bacteria, or about 10⁸ to about 10²⁰ attenuated bacteria, or at least about 10²⁰ attenuated bacteria.

### Modes of Administration

The vaccine compositions of the present invention may be administered intranasally, intradermally, via cloaca, intravenously, intraperitoneally, subcutaneously, orally, by aerosol (spray vaccination), in drinking water, in feed or intramuscularly. For application to poultry, administration by aerosol and eye-drop administration are preferred.

Spray-vaccination is particularly preferred as it allows the vaccination of large numbers of subjects by simply nebulising or creating an aerosol of the vaccine composition in the presence of the subjects to be vaccinated. Nebulising the vaccine composition produces an aerosol of the vaccine composition (live attenuated bacteria) when this aerosol is inhaled by the animals the live attenuated virus is delivered to the animal and invades the respiratory tract mimicking a respiratory disease. This method of vaccination is particularly efficient because time-consuming individual handling of the animals is not necessary.

The present invention will now be further described in greater detail by reference to the following specific examples, which should not be construed as in any way limiting the scope.

### EXAMPLES

### Example 1

### Creation and Identification of a M. gallisepticum Mutant Library

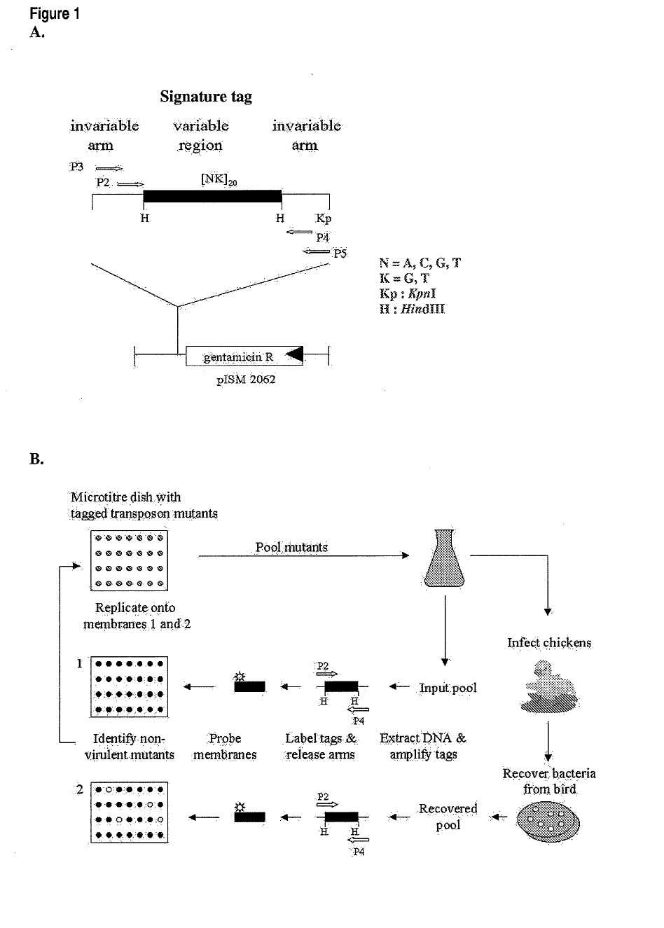

Signature-tagged mutagenesis (STM) was used to generate and identify an M. gallisepticum mutant library. The STM strategy is shown in FIG. 1. Unique DNA tags are incorporated into a transposon, which is used to transform the pathogen, resulting in random insertion of the signature tag in the genome. Occasionally, the signature tag (FIG. 1 A) may integrate into and inactivate a gene, producing an insertional mutation. A negative selection process is then used to screen a pool of ST mutants in a suitable animal model. The input pool and ST mutants recovered from the animal are compared using PCR amplification of the individual tags, followed by hybridisation. The technique identifies individual mutants that are present in the input (pre-selection) pool, but missing from the pool recovered following passage in the animal subject (FIG. 1 B). These missing mutants are most likely to contain mutations in genes responsible for colonisation and growth in the subject and therefore these genes are likely to encode virulence-related determinants.

### Materials and Methods

### Bacterial Strain and Cultural Conditions

M. gallisepticum (Mg) Ap3AS was originally isolated from the air sacs of a broiler chicken in Australia and is highly pathogenic. It was grown in modified Frey's broth (MB) containing 10% swine serum at 37° C. until late logarithmic phase (pH approximately 6.8).

### Signature-Tagged Transposons

An ST mutant library was prepared using the plasmid pISM 2062.2 carrying the transposon Tn4001mod, which contained the gentamicin resistance gene. Each signature tag consisted of a unique 40 by oligonucleotide DNA sequence ([NK]20; N=A, C, G or T; K=G or T) flanked by two invariable arms of 20 bp that enable the amplification and labeling of the unique regions by PCR using the primer pairs P2/P4 or P3/P5. The signature tag was cloned into the KpnI restriction site of pISM 2062.2 (FIG. 1 A). The sequences of the individual signature tags are listed in Table 1.

TABLE 1
DNA sequences of signature tags used in this study

| | Original | | SEQ | Size | |
|---|---|---|---|---|---|
| Tag ID | ID | DNA sequence* | ID No | (bp) | P2/P4^{#} |
| Tag 01 | Ptag5 | CCACAGCGCTTTCTCCCGCGCCCAATCACCCACCCTCAAT | 1 | 40 | 80 |
| Tag 02 | Ptag7 | AGCTACAAGCGAACTACCTCCCGCTATCACCCGCTCGCA | 2 | 39 | 79 |
| Tag 03 | Ptag9 | ACCCATACCGAAACAACTCTCCCGCAAGCGCCCAACCCCT | 3 | 40 | 80 |
| Tag 04 | Ptag10 | ATCGAGGTATCGGTTGCTAGGGGGTACGCTAGGGGGAGTT | 4 | 40 | 80 |
| Tag 05 | Ptag12 | same as Tag 17 (R3-9) | 5 | | |
| Tag 06 | Ptag17 | AGAGCTTGAAATATTGGGCGCGAGTGTTCAGGGTTGGGGT | 6 | 40 | 80 |
| Tag 07 | Ptag20 | AGATGAGGGGCTGGAGCTAGAGATGCTGATTTAGCGGAGG | 7 | 40 | 80 |
| Tag 08 | 1R1-5 | same as Tag 04 (Ptag 10) | 4 | | |
| Tag 09 | 1R1-10 | ATCTATCCACCGATACTCCTCTCAATCTAGCGCGATACCT | 8 | 40 | 80 |
| Tag 10 | 1R1-20 | GTCGTGCGTGGGAGAGGGCTATGGATGGTTGATGAGGGGT | 9 | 40 | 80 |
| Tag 11 | 1R1-29 | CTATATCTACATCTCTCACCCGAGCGCTCCCCAAACCGCC | 10 | 40 | 80 |
| Tag 12 | R2-3 | CTCCACCTCTATCTCTCTATCCCCACATAGACCCACCTCC | 11 | 40 | 80 |
| Tag 13 | R2-5 | CCTATCACGCTTCCTCCTTCAACCTAACTCCCACTAGAGCT | 12 | 41 | 81 |
| Tag 14 | R2-9 | ATCTCCCTATCTCCCTCGAGCTCTTGCCCTCTAACAAGCT | 13 | 40 | 80 |
| Tag 15 | R2-18 | GTAATTCGTTCGGTATTTATTGGTCTGGCGGTAGGTAGTAG | 14 | 41 | 81 |
| Tag 16 | R2-22 | CTCCCTCTACTCCCCCGATACACACCCCTACCCACCAATCC | 15 | 41 | 81 |
| Tag 17 | R3-9 | CTGGGGGTGGAGAGGTCGGTGATTAGGAGTCTCGGTGTTA | 5 | 40 | 80 |
| Tag 18 | R3-17 | AAACCCCTGCCACGTCAGCGATCTCCCTCACACTAGCTCT | 16 | 40 | 80 |
| Tag 19 | R3-23 | CTCCCATTCCCACTCACCCGATCTTTGCCACTCCAAACCC | 17 | 40 | 80 |
| Tag 20 | R3-24 | ACTACGACATATCCTACACTACAACGCTACATCCCGCGCT | 18 | 40 | 80 |
| Tag 21 | R3-33 | GGAGATGTTCAAGTTAGGGAGGTGGCTGTAGGTAGAGAG | 19 | 39 | 79 |
| Tag 22 | R3-35 | AKCGGTATGTCGGTAGAGTGGTTGGGGTAGGAGGAGAGCG | 20 | 40 | 80 |
| Tag 23 | R3-47 | AGAGCGGGATAGTGTAGTTATCGTTAGGTCTTGTGGTTTTA | 21 | 41 | 81 |
| Tag 24 | R3-50 | AGAGATTTATCGATAGCGAGGTCGTGCTGGCGAGTGGGTG | 22 | 40 | 80 |
| Tag 25 | R3-54 | GTCGGGAGGGAGAAGATCAGTGTGTTG | 23 | 27 | 67 |
| Tag 26 | R3-57 | AGAGAAGTATCGGGATGTCGCTCGCGCTTGTGTGGGGGAA | 24 | 40 | 80 |
| Tag 27 | R3-66 | GTATGGGTAGGGGGAGAGGGAGCTAGTGATTTACAGGGAT | 25 | 40 | 80 |
| Tag 28 | R23 | GGCGGGGTGTAGAGAGATTGGGAGGGATGTACAGCGGGTT | 26 | 40 | 80 |
| Tag 29 | R26 | CGGGAGATAGAAGTTTTGAGAGGGAGAGTTAGATTGAGTG | 27 | 40 | 80 |
| Tag 30 | R32 | GGTGTGGGCGCTTAGTTGGGCAAGCTTGCGTGGTGGGTCT | 28 | 40 | 80 |
| Tag 31 | R43 | AGGTAGATATCTATAGAGGTAGTTAGGCTGGGGATCGAGGA | 29 | 41 | 81 |
| Tag 32 | R46 | CTCTATCCATCTCGTAACCCATATCGATAGATCGCAATAC | 30 | 40 | 80 |
| Tag 33 | R52 | CGATACAGACCCGAAATCTCTCAACCCCGCACGCAAACT | 31 | 39 | 79 |
| Tag 34 | R56 | ATCTAGCACCAACACTATCCATAGCCCACTATATATATTC | 32 | 40 | 80 |
| Tag 35 | R62 | GGGGGTAGAGCGTGAGCGGGATGTGTCGGGGGGTAGGGAG | 33 | 40 | 80 |
| Tag 36 | R75 | AAAGCTCTGGTGTCTGGGCGCTAGGGAGGGATCGAGGAGG | 34 | 40 | 80 |
| Tag 37 | R77 | CCCACACTCTCCGACCCCTCAGATCCCCCCCGCCAACCCT | 35 | 40 | 80 |
| Tag 38 | R79 | AGGGCGAGATGTAGGTGTATGGAGCTGGGGATGGAGATAA | 36 | 40 | 80 |
| Tag 39 | R80 | CCAACTCCTCCGTACACCCCCGCTAGCACCCCAATCCCTCG | 37 | 41 | 81 |

*DNA sequence of unique oligonucleotide without the invariable arms
^{#}P2/P4 primer set were generate from the invariable arms of signature tags.
The expected size (bp) of products after PCR amplification are indicated.

### Signature-Tagged Mutagenesis

Transformation with Signature Tags

Dilutions of Mg Ap3AS were grown in 1.5 ml of MB overnight at 37° C. The dilutions showing a colour change were combined and the cells pelleted by centrifugation for 5 min at 16,000 g at room temperature. The cells were resuspended in 200 μl of chilled HEPES-sucrose buffer (8 mM HEPES, 272 mM sucrose (pH 7.4)). The cells were pelleted by centrifugation for 5 min at 16,000 g at room temperature and washed a further two times with chilled HEPES-sucrose buffer, then finally resuspended in 400 μl of HEPES-sucrose buffer chilled to 4° C.

A 100 μl aliquot of cells was placed on ice and 10 μg of plasmid DNA was added. The cell-plasmid DNA mixture was then transferred to a chilled 0.2 cm gap cuvette (Bio-Rad) on ice and immediately electroporated using a Gene Pulser™ (Bio-Rad) with the settings of 2.5 kV, 100 S2 resistance and 25 μF capacitance. The electroporated cells were then gently resuspended in 1 ml cold (4° C.) MB and incubated at room temperature for 10 min, followed by incubation at 37° C. for 2-3 hours, depending on whether there was a colour change. A 500 μl sample of the electroporated culture was spread on a MA plate containing gentamicin at 160 μg/ml by gentle tilting and the excess removed using a Pasteur pipette. The plate was then dried and incubated at 37° C. for 7-10 days in an air-tight jar.

### Confirmation of ST Mutants

Mg colonies growing on plates were selected using a Pasteur pipette, placed in 1 ml of MB containing gentamicin (160 μg/ml) and this culture incubated until the media showed a colour change. The cells from a 200 μl volume of the culture were pelleted by centrifugation in a microfuge tube at 16,000 g for 5 min at room temperature and the pellet resuspended in one tenth of the original volume of distilled water. The cells were then heated at 100° C. for 5 min and 2 μl from this used as template for PCR. To confirm the presence of a ST transposon, the oligonucleotide primers P2 and P4 (Table 2) were used to amplify the region within the invariable arms that contained the unique sequence (FIG. 3). The PCR was conducted in a 20 μl reaction volume and contained 2 μl of 10× reaction buffer, 1 μM of each primer, 200 μM of each dNTP, 1.5 mM MgCl₂ 1.5 U of Taq DNA polymerase (Promega) and 2 μl of template DNA. The reactions were performed in a thermocycler (Omnigene, Hybaid) with one cycle at 98° C. for 2 min, followed by 35 cycles of 94° C. for 30 s, 50° C. for 30 s and 72° C. for 40 s, and a final incubation at 72° C. for 7 min. The PCR products were electrophoresed in a 2% agarose gel along with size standards (pUC18 digested with HaeIII).

TABLE 2
Oligonucleotides used in STM studies

| | | SED | |
|---|---|---|---|
| Name | Use | ID No. | 5-3′ sequence (size in bp) |
| P2 | Signature tag region PCR | 38 | ATCCTACAACCTCAAGCT (18) |
| P4 | Signature tag region PCR | 39 | ATCCCATTCTAACCAAGC (18) |
| IGstmGenmeF3 | DNA sequencing | 40 | GGACTGTTATATGGCCTTTTTGGATC (26) |
| | ST mutant PCR | | |
| MG-F | Mg 16S gene PCR | 41 | GTTGCAAATCCGTAAGGTGG (20) |
| MG-R | Mg 16S gene PCR | 42 | TTAGCAACACGGTTTTAGAT (20) |
| EOGentSpelRev | Gentamicin gene PCR | 43 | actagtATCAGCCAATCGCTTAATTG (26) |
| EPGentSpelFor | Gentamicin gene PCR | 44 | actagtCTGAGTTTATGGAAGAAGTT (26) |
| M13-For | DNA sequencing | 45 | GTTGTAAAACGACGGCCAGT (20) |
| M13-Rev | DNA sequencing | 46 | CAGGAAACAGCTATGACC (18) |
| STM02-B-Rev | ST mutant PCR | 47 | TTCAATTCGAAAAATGAGTT (20) |
| STM03-B-Rev | ST mutant PCR | 48 | ACAGCTTGACGTTTTCCA (18) |
| STM04-B-1 | ST mutant PCR | 49 | CGGGGACACAGTAAGGCTAA (20) |
| STM04-C-Rev | ST mutant PCR | 50 | CGCATTGATTCCTACCAC (18) |
| STM06-A-Rev | ST mutant PCR | 51 | AACATGGATTAGACGTTTGC (20) |
| STM09-B-Rev | ST mutant PCR | 52 | TGCCTTTCTTAATAGTGCTCA (21) |
| STM10-Rev | ST mutant PCR | 53 | TTGTGCCCATTCGTTAGT (18) |
| STM13-JA-rev | ST mutant 13 sequencing | 54 | TTAATACGCCTGGGTAAGGTCG (22) |
| STM13-JB-for | ST mutant 13 sequencing | 55 | TATCTATCACGTAATTGGAATGC (23) |
| STM13-KE | ST mutant 13 sequencing | 56 | TCGCTATAACATTTGATTTAG (21) |
| STM13-KE-C′ | ST mutant 13 sequencing | 57 | CATTTTGATGTTTGCGTCA (19) |
| STM13-KE-C′-1 | ST mutant 13 sequencing | 58 | CTTCAAGTTCTCTGTGG (17) |
| STM13-KE-C′-1-Rev | Wild-type Ap3AS PCR | 59 | CCACAGAGAACTTGAAG (17) |
| STM13-KF | ST mutant 13 sequencing | 60 | CCGTTTTTCTTTTATTCGTATTATC (25) |
| STM13-KF-C′ | ST mutant 13 sequencing | 61 | TATAAACCTGGTACGG (16) |
| | Wild-type Ap3AS PCR | | |
| STM13-KF-C′-1 | ST mutant 13 sequencing | 62 | CACACTGAAAGTTATATTA (19) |
| STM13-KF-C′-2 | ST mutant 13 sequencing | 63 | CTCACTATATAAAATAGC (18) |
| STM13-KG | ST mutant 13 sequencing | 64 | CAGAAGATAAGTCTTTAACAG (21) |
| JX-STM17 | ST mutant PCR | 65 | CTAGAAGAAGAAGAAGAGGTAACGAAGC |
| STM18-Rev | ST mutant PCR | 66 | AGCAAAATTTCCACCCAAGA (20) |
| STM20-Rev | ST mutant PCR | 67 | GAGACATCAATCGCGGTTTT (20) |
| JZ-STM22 | ST mutant PCR | 68 | CTCAATTGAAGAATTATATGATG (23) |
| STM23-B-Rev | ST mutant PCR | 69 | TTAACCCCATTCTTGCAG (18) |
| STM26-Rev | ST mutant PCR | 70 | CATCCCACCTGAAAACTCGT (20) |
| | Wild-type Ap3AS PCR | | |
| STM26-genome | Wild-type Ap3AS PCR | 71 | GATCGGTGCACAGATTAAAG (20) |
| KB-STM33 | ST mutant PCR | 72 | ACTACACGGTAGTTAGTAC (19) |

Lower case indicates nucleotide modifications to produce a restriction endonuclease cleavage site

### Determination of Transposon Insertion Points

Isolation of DNA from ST Mutants

Each ST transformant was grown in 40 ml of MB supplemented with 160 μg of gentamicin/ml at 37° C. until late logarithmic phase (pH approximately 6.8). The cells were harvested by centrifugation at 20,000 g for 30 min and washed twice in chilled PBS, SDS added to lyse the cells and the solution then passed through a 26 gauge needle to shear the genomic DNA. Genomic DNA extraction was performed with the HighPure PCR kit (Roche) according to the manufacturer's protocol, with minor modifications. The initial lysozyme treatment was omitted and the DNA was eluted from column in a volume of 500, rather than 2000, of 10 mM Tris buffer (pH 8.0). The amount of purified DNA was estimated by electrophoresing a 1 μl Sample in a 0.7% agarose gel together with molecular weight standards of known concentration (phage λ DNA digested with HindIII).

### Genomic DNA Sequencing

The procedure for genomic DNA sequencing was adapted and modified from Wada (2000). The primer IGstmGenmeF3 (Table 2), which binds to Tn4001, was used for sequencing across the transposon-genomic DNA junction and into the Ap3AS genomic DNA. The sequence was determined using ABI PRISM Big Dye 3.1 Terminator chemistry (Applied Biosystems Incorporated). Each reaction consisted of 2-3 μg purified genomic DNA, 10 μM of primer and 4 μl of Big Dye 3.1 enzyme mixture, and cycle sequencing was performed in an iCycler™ (Bio-Rad) using one cycle at 95° C. for 5 min, followed by 60 cycles of 95° C. for 30 s, 55° C. for 30 s and 60° C. for 4 min. The product was then cleaned according to the manufacturer's recommendations and then analysed using an ABI 3100 Capillary Sequencer and relevant software (Applied Biosystems Incorporated). The insertion site for each ST transposon was determined using the BLAST program (National Center for Biotechnology Information, NCBI) or FASTA version 3.3t07 (Pearson and Lipman, 1988) to compare the DNA sequence to that of the M. gallisepticum strain Rlow genome.

### Detection of Mg ST Mutants

Oligonucleotides complementary to each of the 37 signature tags were synthesised. The oligonucleotides were resuspended in TE buffer to a concentration of 100 μM. A 10 μl volume of a solution of each oligonucleotide (containing 3 picomoles) was spotted onto a Hybond-N+ nylon membrane (Amersham Pharmacia Biotech) using the Bio-Dot SF® Microfiltration Apparatus (Bio-Rad) following the manufacturers' instructions, and the oligonucleotides then fixed to the membrane by UV cross-linking for 3 min. DNA probes corresponding to each signature tag were synthesised using the PCR reaction described above except that the oligonucleotide primers P2 and P4 were commercially labelled with digoxigenin (DIG) (Roche). The DIG labelled probes were hybridised to the membranes in DIG Easy Hyb buffer (Roche) for 16-18 hr at 40° C. in a shaking water bath and then the membranes washed according to the manufacturer's instructions, except that the second wash was done at 45° C. Bound probes were detected using the DIG Luminescent Detection Kit (Roche) and detection recorded using Biomax film (Kodak). PCRs were also conducted to confirm the presence of the gentamicin resistance gene and the species of the mycoplasma transformant. Each PCR reaction was performed using 1.5 U Taq DNA polymerase in a 25 μl reaction mix containing 1.5 mM MgCl₂, 200 μM of each dNTP and 1 μM of each primer. A region of the Mg 16S rRNA gene was amplified by incubation at 95° C. for 5 min, then through 35 cycles of 95° C. for 10 s, 58° C. for 10 s and 72° C. for 25 s. The expected product was 219 by in size. The fragment of the gentamicin resistance gene was amplified by incubation at 95° C. for 2 min, then through 28 cycles of 95° C. for 30 s, 60° C. for 30 s and 72° C. for 15 s, with a final incubation at 72° C. for 5 min. The expected size of the product was 278 bp.

### Cross-Hybridisation Between Signature Tags Using the DIG Detection System

A pool of DIG labelled signature tags was hybridised to a single membrane containing 37 signature-tag oligonucleotides as described above. The DIG labelled tags would be expected to bind only to the corresponding unique oligonucleotides unless there was cross-hybridisation between the tags and probes. For example, a pool containing labelled Tags 02, 03 and 04 would be expected to only bind to oligonucleotide Tags 02, 03 04 on the dot blot, and any other positive reactions should be regarded as cross-reactions. Cross-hybridisation reactions were assessed using 13 different pools of labelled signature tags.

### Preliminary Animal Experiment

### Calculation of Numbers of Viable Cells of St Mutants

The viable count of each ST mutant culture, measured as colour changing units per ml (CCU/ml), was determined using the following procedure. To each well of a sterile 96 well microtitre plate (Nunculon, Nunc), 225 μl of sterile MB containing gentamicin (160 μg/ml) was added. To the first column of 8 wells, 25 μl of mutant culture was added to each well, mixed several times by pipetting and 25 μl from each well transferred to the next column and mixed using a fresh set of tips. This sequence of serial 10-fold dilutions was repeated until column 10, from which 25 μl was discarded. Columns 11 and 12 were used as negative controls. The plate was then sealed with a Linbro® plate seal (ICN Biochemicals) and incubated at 37° C. for up to 2 weeks. A drop in the pH of the medium reflected growth of the culture, and was indicated by a colour change from red to yellow of the phenol red indicator in the medium. Column 1 was regarded as a 1/10 dilution and after correction for the initial dilutions of the culture, the numbers of CCU/ml were calculated using most probable number tables. The dose of each transformant needed for inoculation of chickens was 1×10⁷ CCU/ml.

### Experimental Design

Sixteen 4-week-old specific pathogen free (SPF) White Leghorn chickens were housed in a positive pressure fibreglass isolator. A series of dilutions of ten ST mutants were grown overnight in a microtitre plate in MB containing gentamicin at 160 μg/ml (Table 3). The growth of each clone was assessed the following day and 100 μl taken from the dilution estimated to be at log phase and added to the pool. The pooled ST mutants were then diluted another tenfold in MB and this diluted pool was used as the inoculum. Twelve of the sixteen chickens were infected with the pooled ST mutants by administration as an aerosol. The remaining 4 birds served as in-contact controls and were placed in the isolator 3 days after aerosol infection. Six of the aerosol infected birds were euthanased and sampled 14 days after infection and the remainder, including all the in-contact birds, 28 days after infection.

TABLE 3
Transposons tested

| ST mutant | % of gene to insertion point |
|---|---|
| 02-1 | (68.3) |
| 03-1 | (16.0) |
| 04-1 | (0.2) |
| 06-1 | 54.6 |
| 09-1 | 60.3 |
| 17-1 | 80.1 |
| 19-1 | 88.6 |
| 32-1 | 52.0 |
| 35-1 | 76.4 |
| 37-1 | 20.5 |

### Collection and Analysis of Samples

Blood samples were collected from all birds before aerosol infection and before euthanasia and left to clot at room temperature for 3 h. Serum was then harvested by centrifuging the sample at 10,000 g for 5 min at room temperature and then tested using the RSA test to determine antibody responses against M. gallisepticum. The RSA test was performed by mixing 25 μl of undiluted serum and 25 μl of stained commercial Mg RSA antigen (Intervet International) on a glass plate and rocking for 1 min. The test was scored using a scale from 0 (no clumping) to 4 (large clumps of antigen). Each test included both a negative serum and a positive serum as controls. Chickens were euthanased by carbon dioxide inhalation. The gross air sac lesions were examined and scored for severity on a scale of 0 to 3 as follows:
0=no lesions, air sacs a thin clear film
1=small flecks of caseous fibrin attached to the air sacs.
2=air sacs appear cloudy and thickened, with moderate amounts of caseous fibrin attached to the air sacs.
3=thickened air sacs and surface covered with thick caseous fibrin.

A half score was given for lesions that lay between these definitions. A score was recorded for the left and right anterior thoracic air sacs, the left and right posterior thoracic air sacs and the left and right abdominal air sacs. The sum of the six scores was used as the final score for each individual bird. Swabs were taken from the air sacs, trachea, lung, spleen, liver, kidney and brain of each bird. The swabs were used to inoculate MA plates containing 160 μg gentamicin/ml as well as MA plate without gentamicin and were then placed into 3 ml of MB supplemented with gentamicin at 160 μg/ml. MA plates were incubated at 37° C. and examined using a binocular dissecting microscope after 7-10 days. A previous study detected the loss of the Tn4001 transposon from some mutants during in vivo experiments 14 days after inoculation. These mutants regained the wild-type phenotype but could not survive under the selection pressure of gentamicin. When the numbers of colonies on plates containing no gentamicin were tenfold or greater than the numbers of colonies on plates containing gentamicin loss of the transposon was suspected. MB cultures were incubated at 37° C. and DNA was extracted from those broths showing a colour change and used as template in PCRs as described above to amplify the unique tag region using the P2/P4 primer pair.

### Results

### Confirmation of ST Mutants

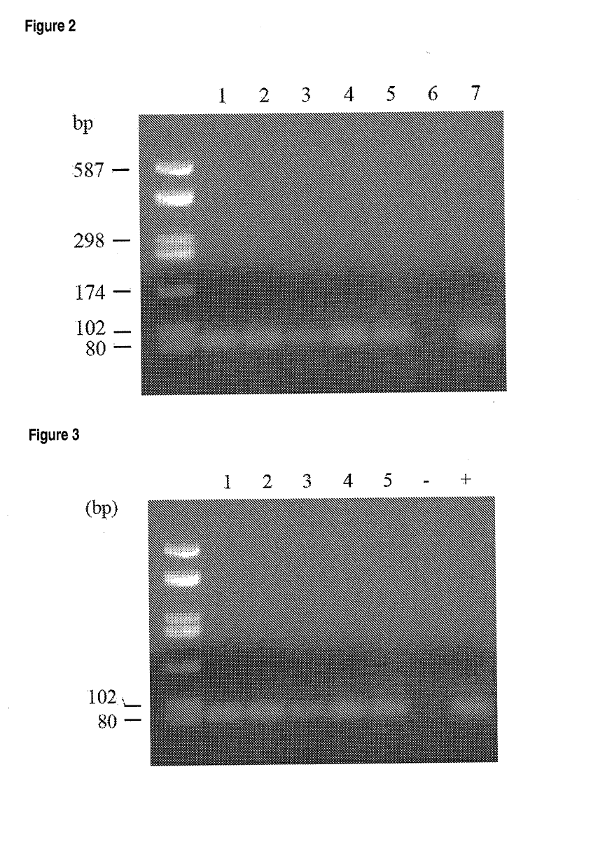

The expected size of the PCR product generated from signature tags using the P2/P4 primer pair was between 79 to 81 bp, with the exception of that from Tag 25, which was 67 bp. Each plasmid containing a signature tag was introduced into Mg strain Ap3AS and the transformants were chosen randomly and grown in media supplemented with gentamicin. All MB cultures showing growth were subjected to PCR to detect the presence of an ST mutant containing a unique signature tag (FIG. 2). The ST mutant library was established with at least 9 transformants generated from each signature tag and used for further experiments.

### Determination of Transposon Insertion Site

The transposon insertion site for each of the ten Mg ST clones used in the studies described in this chapter was determined by directly sequencing genomic DNA (Table 3). The transposons had inserted into regions encoding either unique or conserved hypothetical proteins in five ST mutants.

### Optimised Detection of ST Mutants

### Cross-Hybridisation Between Signature Tags

Most tags were specific, with only a few cross hybridising with oligonucleotides complementary to other tags. There was a strong cross-reaction between Tags 02 and 37 (in pools A, E, G and H). Some cross hybridisation was also detected between Tags 15 and 30 (in pools D, I and J), but no cross-hybridisation was detected with pool B. There was one-way hybridization between Tag 13, when it was in the pool, and Tag 28. This was also seen with Tags 29 and 23 (pools D and H), and Tags 29 and 24 (pools D and H).

Preliminary Assessment of Infection of Chickens with ST Mutants

### Serological Testing and Gross Examination of Air Sacs

Anti-M. gallisepticum antibody responses were determined by RSA (Table 4) before and after infection. No M. gallisepticum specific antibody was detected in birds before infection. Of the chickens infected by aerosol, two thirds had RSA scores greater than one at 2 weeks after infection and all were positive by 4 weeks after infection, with RSA scores between 1 and 4. Only one of the in-contact controls had a RSA score of 1. Mild air sacs lesions (score of 0.5) were seen in 2/6 birds 2 weeks after infection and in 3/6 chickens at 4 weeks after infection, while one bird had severe lesions in the abdominal air sacs (score of 2.5). Only one of the in-contact controls had mild lesions (score of 0.5). Detailed results are shown in Table 4.

TABLE 4
ST mutants recovered from different sites in birds in the preliminary experiment

| | | | Air sacs | | | | | | | | | | | | | | | | |
|---|---|---|---|---|---|---|---|---|---|---|---|---|---|---|---|---|---|---|---|
| | | | Le- | | | Trachea | | Lung | | Heart | | Spleen | | Liver | | Kidney | | Brain | |
| Time | Bird | RSA | sions | MA | MB | MA | MB | MA | MB | MA | MB | MA | MB | MA | MB | MA | MB | MA | MB |
| Week 2 | 744 | 0 | 0 | − | − | − | − | − | − | − | − | − | − | − | − | − | − | − | − |
| | 745 | 1 | 0.5 | − | − | − | − | − | − | − | − | − | − | − | − | − | − | − | − |
| | 746 | 1 | 0.5 | − | 37* | + | 35, 37 | − | − | + | − | − | − | − | − | − | − | − | − |
| | 747 | 0 | 0 | − | − | − | 32 | − | − | − | − | − | − | − | − | − | − | − | − |
| | 748 | 0 | 0 | − | − | + | 32 | − | − | − | − | − | − | − | − | − | − | − | − |
| | 751 | 0 | 0 | − | − | − | 32, 37 | − | − | − | − | − | − | − | − | − | − | − | − |
| Week 4 | 741 | 1 | 0 | − | − | − | − | − | − | − | − | − | + | − | − | − | − | − | − |
| | 742 | 1 | 0 | − | − | − | − | − | − | − | + | − | + | − | − | − | + | − | 35 |
| | 743 | 2 | 0.5 | − | 32 | + | 32 | − | − | − | − | − | + | − | − | − | − | − | − |
| | 749 | 4 | 2.5 | + | − | + | − | − | − | − | + | − | − | − | + | − | + | − | − |
| | 750 | 1 | 0 | − | − | + | 35 | − | − | − | + | − | + | − | − | − | + | − | − |
| | 757 | 2 | 1.0 | − | 37 | + | 37 | − | − | − | − | − | − | − | + | − | − | − | − |
| In- | 737 | 0 | 0 | − | − | − | − | − | − | − | − | − | − | − | − | − | + | − | + |
| contact | 738 | 0 | 0 | − | contam. | − | 02 | − | − | − | − | − | − | − | − | − | + | − | − |
| controls | 739 | 0 | 0 | − | − | − | − | − | − | − | − | − | − | − | − | − | − | − | − |
| (Week | 740 | 1 | 0.5 | − | − | − | − | − | − | − | − | − | − | − | − | − | − | − | + |
| 4) | | | | | | | | | | | | | | | | | | | |

RSA: Rapid serum agglutination
contam: contaminated culture
*Detected tag ID by hybridisation and positive by PCR for the presence of the gentamicin gene and Mg 16s rRNA gene.
+: indicates Mg colonies grew on MA or a colour change was seen in MB.
−: no growth seen on MA plate or in MB

### Reversion and Identification of Recovered ST Mutants

MA plates were examined for growth of M. gallisepticum in the presence and absence of gentamicin. There was no evidence of the loss of the transposon from ST mutants after in vivo passage. M. gallisepticum was only isolated on MA plates inoculated with swabs of the air sacs of one bird and the tracheas of two birds at 2 weeks after inoculation, and of the tracheas of four chickens at 4 weeks after infection. M. gallisepticum was also isolated from a swab taken from the heart of one bird 14 days after infection. No M. gallisepticum was isolated from swabs of any organ of the in-contact controls (Table 4). One broth culture of a swab of the air sacs taken at 2 weeks after infection and four of the tracheas showed a colour change with 3 of the 10 unique tags detected by DIG hybridisation from these samples. Broth cultures showing a colour-change were obtained from swabs of the air sacs of two birds and swabs of the tracheas of three birds at day 28 after infection, as well as from swabs of other organs, including the lungs of five birds, the hearts of four birds, the livers of two birds, the kidneys of three birds, the spleens of four birds and the brain of one bird. Broth cultures showing a colour change were also obtained from swabs of the tracheas of one, the kidneys of two and the brains of two of the in-contact birds. However, PCRs conducted to detect the presence of the gentamicin resistance gene and the Mg 16S rRNA gene in cultures showing a colour change after 21 days incubation were all negative, suggesting that the colour change observed in these cultures did not result from growth of M. gallisepticum. The mutants carrying Tags 02, 32, 35 and 37 were truly recovered from the air sacs, tracheas of the birds and from brain of one bird with the most commonly detected tags being Tags 32 and 37 (Table 4). Several broth cultures showed a colour change within 7 days after incubation, but tags could not be detected by DIG hybridisation (data not shown).

### Example 2

### In Vivo Analysis of M. gallisepticum ST Mutants

### Introduction

The STM technique consists of two major steps: creation of a tagged mutant library in vitro and then negative selection in vivo in an animal. Using the modified hybridisation detection system described in Example 1 above, the input pool contained only mutants derived from tags that did not cross hybridise with each other and also yielded clear signals for detection. The ST mutant library of Example 1 was generated using 37 different tagged transposons. This example describes the screening of 102 ST mutants containing 34 different tags to identify genes involved in virulence or required for survival in vivo using negative selection in infected chickens.

### Materials and Methods

### Determination of Insertion Sites in ST Mutants

Genomic DNA from each ST mutant was subjected to direct sequencing using a specific primer (Table 2). For those ST mutants from which sequence data could not be obtained, or which generated mixed sequencing signals after the end of the transposon, Southern blotting was used to determine the number of transposon insertions. The ST mutants with more than one transposon insertion were then omitted from further attempts to identify the transposon insertion points. The probes for Southern blotting were prepared using DIG-labelled primers (P2 and P4) to amplify the tag region (Table 2). The genomic DNA of the ST mutant was digested overnight with 20 U of the restriction endonuclease BglII (New England Biolabs) at 37° C. and the is resultant fragments separated in a 0.7% agarose gel overnight at 2.5 V/cm. The separated fragments were then transferred onto a Hybond N+ nylon membrane hybridised to the DIG-labelled probe and hybridisation detected using the DIG Luminescent Detection Kit (Roche) according to the manufacturer's instructions and luminescence recorded on Biomax Film (Kodak).

### Screening of ST Mutants in Infected Birds

### Preparation of ST Mutants

A ten-fold dilution series of each of the 34 ST mutants, each of which contained a distinct tag, for each of the three inocula (Groups A, B and C) (102 mutants in total for the three experimental groups) was made in microtitre plates in MB supplemented with gentamicin (160 μg/ml) and incubated at 37° C. overnight (Table 5). The dilution of each mutant that had produced an appropriate colour change was used for inoculation. A 0.1 ml sample of the appropriate culture of each mutant was taken and mixed with the cultures of the other mutants to create a pooled inoculum. The pooled ST mutants were then used immediately for aerosol inoculation of chickens.

TABLE 5
Transposon insertion sites in ST mutants
generated in this study (Inoculum A)

| | Insertion site in genome | |
|---|---|---|
| ST mutant | (% of gene to insertion point) | Function of disrupted gene |
| 26-1 | MGA_0220 (34.2) | ATP-binding protein OppD |
| 26-2 | MGA_0220 (34.2) | ATP-binding protein OppD |
| 26-3 | MGA_0220 (34.2) | ATP-binding protein OppD |

### Initial Screening

### Experimental Design

A total of 90 four-week-old SPF chickens were randomly allocated into 3 groups, each of which was housed separately (30 birds per group) in positive pressure fibreglass isolators. Twenty birds in each group were inoculated with a pool of ST mutants by aerosol, with the remaining 10 chickens acting as in-contact controls, which were placed with the inoculated birds three days after aerosol exposure. In-contact controls were used to investigate the capacity for transmission of the ST mutants. At day 14 after infection, blood samples were collected from ten inoculated chickens and they were euthanased and examined for lesions. The blood samples were tested for antibodies against M. gallisepticum using the RSA test, as described below. All birds were examined for gross air sac lesions and these were assessed for severity on a scale of 0 to 3 as described below. Swabs of the air sacs and trachea of each bird were cultured to recover mycoplasmas as described below. DNA was extracted from each broth culture that changed colour and used as template in PCRs to amplify the unique tag region. The PCR products were then used in Southern blots to identify the ST mutants that were present, as previously described below. The MA plates were checked after incubation for 10 days, as described below. The remaining birds were euthanased, examined and sampled using the same procedures at 28 days after inoculation.

### Confirmatory Screening

Preparation of M. gallisepticum ST Mutants

Dilutions of the ST mutants that were not re-isolated in the initial screening experiment were grown in MB containing 160 μg gentamicin/ml at 37° C. overnight. The dilution of each ST mutant that had caused the appropriate degree of colour change was used for inoculation. A 0.1 ml sample of the culture of each mutant was mixed with samples of the other mutants and this pool then diluted tenfold in MB for use as a new pooled inoculum, which was used to inoculate chickens by aerosol.

### Experimental Design

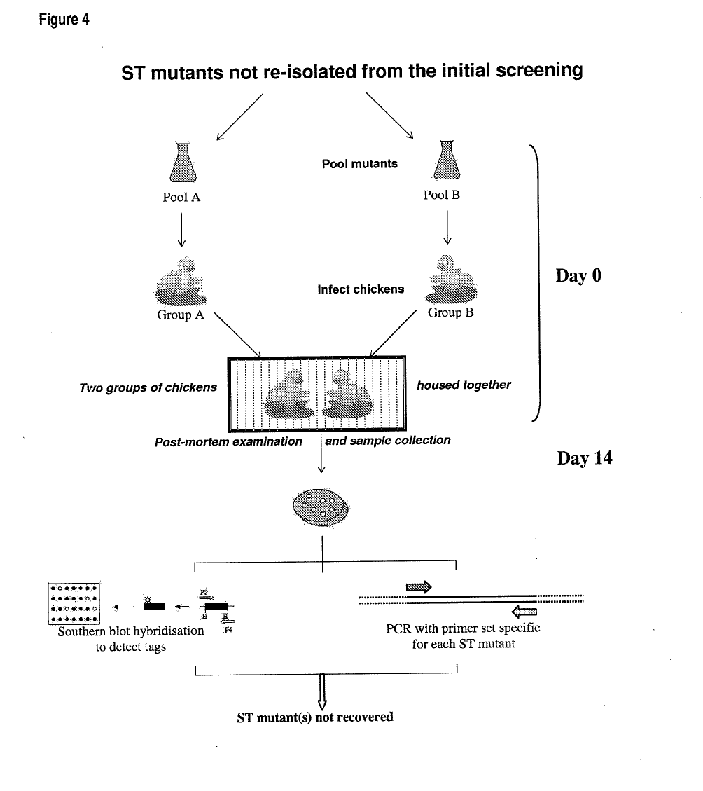

The confirmatory experiment was conducted to narrow down the number of candidate ST mutants for definitive testing. The experimental design and methods were similar to the initial screening experiment, which is shown in FIG. 4. A total of forty 4-week-old SPF chickens were allocated into two groups. The ST mutants that could not be detected in inoculated birds in the initial screening experiment were allocated to one of two inocula, each of which was used to inoculate one of the two groups of the birds by aerosol. In-contact controls were not included in this experiment. However, all 40 birds were placed into the same positive pressure fibreglass isolator and thus each group was able to be used as an in-contact control for the other group. Sample collection was performed 14 days after inoculation using the is same procedures previously described below. Briefly, blood samples were collected prior to inoculation and just prior to euthanasia. Swabs from the air sacs and the trachea were collected at post mortem for isolation of mycoplasmas. All birds were examined for gross air sac lesions. Sera were tested using the RSA assay. DNA was extracted from each broth culture that changed colour and used as template in PCRs to amplify the unique tag regions, which were then used as probes in dot blot hybridisation assays. The MA plates were checked after incubation for 10 days at 37° C., as previously described below. Several colonies were collected from MA plates and cultured in MB supplemented with gentamicin at 37° C. until a colour change was seen. The DNA was extracted and used as template in PCR assays to confirm the species of mycoplasma by amplifying a 219 by fragment of the Mg 16S rRNA gene, as previously described in Example 1 (FIG. 4).

PCR Confirmation of Specific ST Mutants Recovered from Infected Birds

PCR primers were designed that were specific for each ST mutant based on the sequencing results (Table 2). They were used in PCR assays with primer IGstmGenmeF3 to detect each of the ST mutants in cultures from inoculated birds. PCRs were conducted in 20 μl volumes containing 2 μl extracted DNA as template, 2 μl of 10× reaction buffer, 1 μM of each primer, 200 μM of each dNTP and 1.5 U of Taq DNA polymerase (Promega). PCRs were performed in a thermocycler (Omnigene, Hybaid), with one cycle at 95° C. for 2 min, followed by 35 cycles of 94° C. for 45 s, 52° C. for 45 s and 72° C. for 1 min, and a final incubation at 2° C. for 7 min.

### Results

### Mapping the Insertion Points in ST Mutants

The insertion site of the transposon was determined in 91 ST mutants using the direct genomic sequencing technique. Reliable sequence data could not be obtained for 11 mutants (Table 5). In most cases the insertion site was the same in Groups A, B and C, the only exceptions being ST mutants 01-1, 02-2, 02-3, 04-3, 09-3 and 17-3.

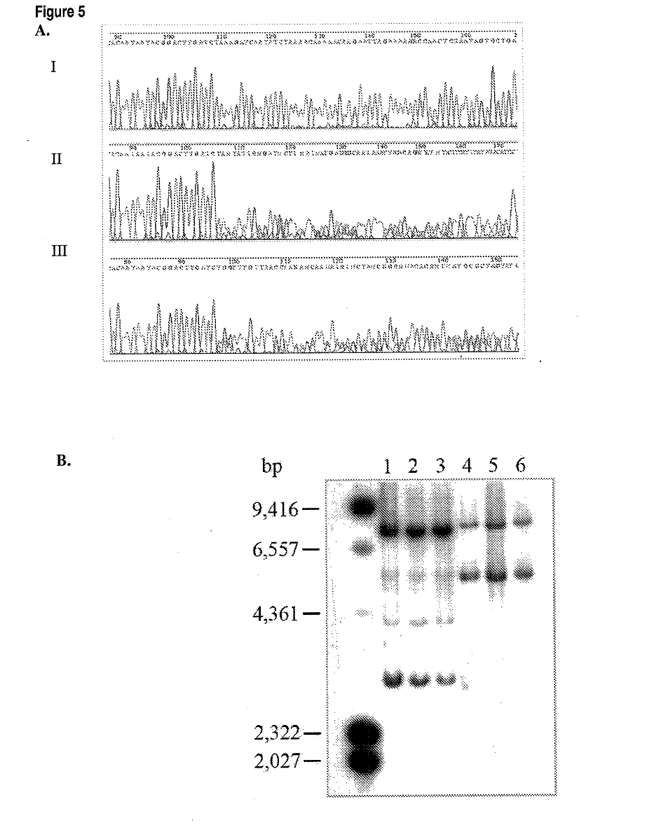

Those ST mutants that could not be sequenced directly were subjected to Southern blotting to determine the number of transposon insertions. The sequencing chromatographs showed that the first 100 by of data, which corresponded to the transposon, were of good quality, but that mixed signals were obtained after the junction of the transposon and genome sequence was reached (FIG. 5 A). As the restriction endonuclease BglII was not predicted to cleave within the transposon sequence, the number of bands bound by the probe should reflect the number of times that the transposon was present within the genomes of the ST mutants. Multiple transposon insertions were confirmed in ST mutants 15-1, 15-2, 15-3, 25-1, 25-2, 25-3, 34-1, 34-2, 34-3, 09-3 and 17-3 by Southern blotting and the mutants carrying the same signature tag appeared to have identical insertions (Tables 5 and 6, FIG. 5 B).

### Initial Screening Experiment

### Pathological and Serological Findings

The RSA results from each experimental group are shown in Table 7. No anti-mycoplasma antibodies were detected in the serum from any bird at the time of inoculation. Generally, more sera were positive at 4 weeks than at 2 weeks after infection, and no antibody response was detected in in-contact birds in any group. More birds had air sac lesions at 2 weeks (2, 2 and 4 in Groups A, B and C, respectively) than at 4 weeks (0, 1 and 2 in Groups A, B and C, respectively) after inoculation. More severe air sac lesions were observed at 2 weeks after inoculation (scores of 0.5 in Group A, 1.0 to 2.0 in Group B and 0.5 to 2.5 in Group C) except that in Group C one chicken had a lesion score of 3.0 at 4 weeks after inoculation. More air sac lesions were found in Group C. The severity of lesions did not correlate well with the RSA results, with some birds having high RSA scores but no detectable air sac lesions.

TABLE 6
Classification of transposon insertion
sites in ST mutants in this study

| ST mutant | Transposon insertion site | Function |
|---|---|---|
| 26-1/-2/-3 | MGA_0220 | ATP-binding protein OppD |
| 09-3 | Multiple insertions | |
| 13-1/-2/-3 | No match | |
| 15-1/-2/-3 | Multiple insertions | |
| 17-3 | Multiple insertions | |
| 25-1/-2/-3 | Multiple insertions | |
| 34-1/-2/-3 | Multiple insertions | |

TABLE 7
RSA results, air sac lesion scores and tags detected in each experimental group in the initial screening

| | RSA* | | | | Air sac lesions* | | | Tags detected (number of times detected) | | |
|---|---|---|---|---|---|---|---|---|---|---|
| Group | Week 0 | Week 2 | Week 4 | Control | Week 2 | Week 4 | Control | Air sacs | Trachea | Undetected\hat{ } |
| A^{#} | 0/30 | 7/10 | 8/9 | 0/8 | 2/10 | 0/9 | 0/8 | 16(1), 20(2), 28(2) | 07(3), 11(1), 12(4), 14(1), | 02 (Group A, B), |
| | | | | | | | | | 18(1), 19(2), 20(6), 21(1), | 03, 04, 06, 09, 10, |
| | | | | | | | | | 24(6), 26(1), 27(2), 28(2), | 15, 17, 22, 23, 25, 33, |
| | | | | | | | | | 31(1), 32(1), 35(1), 38(1), | 34 |
| | | | | | | | | | 39(1) | |
| B | 0/31 | 6/10 | 9/11 | 0/10 | 2/10 | 1/11 | 0/10 | 12(1), 20(1), 28(1) | 07(1), 11(1), 12(3), 14(1), | |
| | | | | | | | | | 16(1), 20(6), 27(1), 28(2), | |
| C^{#} | 0/29 | 5/10 | 9/10 | 0/7 | 4/10 | 2/10 | 0/7 | 02(1), 11(1), 12(1), | 01(1), 02(8), 11(1), 12(5), | |
| | | | | | | | | 16(3), 18(1), 20(7), | 13(1), 14(1), 16(3), 18(1), | |
| | | | | | | | | 27(1), 28(1) | 20(10), 24(5), 27(6), 28(5), | |
| | | | | | | | | | 36(1), 38(2) | |

*Number positive/number examined. Complete RSA and air sac lesion score data are shown in Appendix C.
^{#}One inoculated and two in-control birds died in Group A and two in-contact birds died in Group C prior to post mortem.
\hat{ }Summary of the undetected signature tags of the three groups.

### Identification of Recoverable ST Mutants

The results for broth cultures taken from birds at 14 and 28 days after inoculation are summarised in Table 7. The pattern of recovery of M. gallisepticum was similar to that seen in the preliminary experiment, with more isolations made from the tracheas than the air sacs. Generally, more isolations were made at 2 weeks than at 4 weeks after infection. No ST mutants were re-isolated from the in-contact birds in Group B, although five different ST mutants were re-isolated from the in-contact birds in Group A and two from those in Group C. The ST mutant carrying Tag 20 was the most common to be recovered from the air sacs, as well as from the tracheas, and had a high detection rate in all three groups. The next most commonly isolated mutant was that carrying Tag 28. Sixteen ST mutants, represented by 12 different tags, were not able to be re-isolated, including ST Mutants 02-1 (-2), 03-1 (-2/-3), 04-1 (-2), 04-3, 06-1 (-2/-3), 09-1 (-2), 09-3, 10-1 (-2/-3), 15-1 (-2/-3), 17-1 (-2), 17-3, 22-1 (-2/-3), 23-1 (-2/-3), 25-1 (-2/-3), 33-1 (-2/-3) and 34-1 (-2/-3).

M. gallisepticum was not isolated on MA plates inoculated with swabs of the air sacs or the tracheas of any in-contact chickens. M. gallisepticum were isolated on MA plates inoculated with swabs collected 2 weeks after inoculation from the air sacs of three, one and five birds in Groups A, B and C, respectively. They were also isolated on MA plates inoculated with swabs of the tracheas of five chickens in Group C, but not from any birds in Groups A or B. M. gallisepticum were isolated on MA plates inoculated with swabs collected from the air sacs of four, two and six birds, and the tracheas of one, two and seven chickens in Groups A, B and C, respectively, at 4 weeks after inoculation. In almost every case when mycoplasmas were isolated by agar culture they were also isolated by broth culture. No loss of the transposon was detected in any ST mutant.

### Confirmatory Screening Experiment

Sixteen ST mutants that could not be detected in the initial screening experiment were separated into 2 groups. The sequence data on the transposon insertion site and Southern blotting indicated that the transposon in ST mutants 04-1 and 04-2 had the same insertion site but a different insertion site to that of 04-3. Transposon insertion sites in ST mutants 09-1 and 09-2 were identical, whilst mutant 09-3 had multiple insertions. The same situation was also found in ST mutants 17-1, 17-2 and 17-3. None of these mutants were able to be detected in the initial screening experiment, so they were placed into two different inocula to enable them to be distinguished. Where there was potential transmission between the groups and these three signature tags were detected by dot blot hybridisation, PCR was able to be used to identify the mutants re-isolated from birds.

### Pathological and Serological Assessments

One bird died in Group A and one in Group B during the experiment. Severe air sac lesions (score of 2.5) were seen in one bird, and mild lesions (0.5 and 1.0) in another two chickens in Group A. One bird had mild air sac lesions (1.0) in Group B. No anti-M. gallisepticum antibody was detected prior to inoculation. At 2 weeks after inoculation, 15/19 chickens in Group A were RSA positive, whilst 14/19 birds in Group B were positive. Detailed results are shown in Table 8.

TABLE 8
RSA results, air sac lesion scores and mutants detected in each experimental group
in the confirmatory screening

| | RSA* | | Air sac lesions* | Mutants detected (number of times detected) | | |
|---|---|---|---|---|---|---|
| Group | Week 0 | Week 2 | Week 2 | Air sacs | Trachea | Undetected\hat{ } |
| A^{#} | 0/20 | 15/19 | 3/19 | 03-1(1), 09-1(1), | 02-2(4), 03-1(2), | 04-1, 33-1 |
| | | | | 10-1(2), 15-1(1), | 04-3(1), 06-1(1), | |
| | | | | 34-1(1) | 09-1(3), 10-1(10), | |
| | | | | | 15-1(1), 17-2(12), | |
| | | | | | 23-1(2), 25-1(1), | |
| | | | | | 34-1(1) | |
| B^{#} | 0/20 | 14/19 | 1/19 | 09-3(2), 17-3(1) | 02-2(1), 03-1(1), | |
| | | | | 23-1(2) | 09-3(14), 10-1(5), | |
| | | | | | 15-1(2), 17-3(10), | |
| | | | | | 22-1(2), 23-1(15), | |
| | | | | | 25-1(3), 34-1(2) | |

*Number positive responses/number examined. Complete RSA and air sac lesions data are shown in Appendix D.
^{#}Birds in Group A were inoculated with the pool containing ST mutants 02-2, 03-1, 04-1, 06-1, 09-1, 10-1, 15-1 and 17-2; birds in Group B were inoculated with the pool containing ST mutants 04-3, 09-3, 17-3, 22-1, 23-1, 25-1, 33-1 and 34-1
One bird died in Groups A and B before post mortem
\hat{ }Summary of the undetected ST mutant of two groups.

### Re-Isolation of ST Mutants

M. gallisepticum was more commonly isolated on MA plates inoculated with swabs taken from the tracheas than from the air sacs in both groups. M. gallisepticum was isolated on MA plates inoculated with swabs of the air sacs of two birds in both groups and from swabs of the 18/19 chickens in Group B.

Five ST mutants were recovered from the air sacs of three chickens in Group A and three were recovered from the air sacs of one bird in Group B. More ST mutants were recovered in MB inoculated with swabs taken from the tracheas than from those taken from the air sacs. A total of eleven ST mutants were re-isolated from chickens in Group A and ten were re-isolated from eighteen birds in Group B.

The most common mutant to be re-isolated was the one carrying Tag 23. This mutant was isolated from the tracheas of 17 birds and from the air sacs of two chickens. The second most commonly isolated mutant was 17-2, which was isolated from the tracheas of 12 birds (Table 8). The ST mutants 02-2, 03-1 10-1 and 15-1, is which were used to inoculate birds in Group A, were re-isolated from Group B, whilst ST mutants 04-3, 23-1 25-1 and 34-1, which were used to inoculate birds in Group B, were re-isolated from Group A (Table 8). The ST mutants 04-1 and 33-1 could not be re-isolated from any bird in either group (Table 8). No loss of the transposon was detected in any ST mutant.

### Example 3

### Infectivity and Virulence of Selected ST Mutants of M. gallisepticum

### Preparation of ST Mutants

Two ST mutants, 04-1 and 33-1, which had not been detected after the initial or confirmatory screening experiments, and five that were detected infrequently (ST mutants 03, 18, 20, 22 and 26) were cultured at 37° C. in MB supplemented with gentamicin at 160 μg/ml until late logarithmic phase. Wild-type Ap3AS was cultured at 37° C. in MB that did not contain gentamicin. The concentration of each strain was adjusted to approximately 1×10⁷ CCU/ml using the method described above.

### Virulence and Infectivity Analysis

### Experimental Design

Eight groups of four-week-old SPF chickens were housed separately (20 birds per group) in positive pressure fibreglass isolators. Each of the six groups were inoculated with a different ST mutant by aerosol exposure. Negative control birds were exposed to MB and positive control birds to wild-type Ap3AS. The birds were euthanased at 14 days after infection and post mortem examinations conducted as described above. Sera and swabs were collected from each bird and for anti-mycoplasma antibody detection and mycoplasma isolation were conducted as described above, with the exception of swabs taken from the Ap3AS infected group, which were inoculated onto MA plates and then placed in MB without gentamicin. DNA was isolated from each broth culture showing a colour change and used as template in PCRs to amplify the unique tag region using the P2/P4 primer pair (Table 2). The PCR products were used as probes in dot blot hybridisations to detect the presence of specific tags, as previously described above. PCRs were also performed using the IGstmGenmeF3 primer and a primer specific for each ST mutant as an additional tool to identify the ST mutants in inoculated birds (Table 2). The upper, middle and lower sections of the trachea were taken from each bird, examined histopathologically and the mucosal thickness measured.

### Detection of Re-Isolated ST Mutants

Dot blot hybridisation and PCR amplification were used to detect each ST mutant in the broth cultures. Dot blot hybridisation could not be used for cultures from the positive control group as wild-type Ap3AS did not contain a signature tag. The dot blot hybridisation technique is described in detail above. Briefly, the tag regions in the DNA extracted from the cultures showing a colour change were amplified using the DIG-labelled primer set. Oligonucleotides corresponding to the seven signature tags that identified the mutants used in the experiment were spotted onto nylon membrane and subsequently used in hybridisations. The DIG Luminescent Detection Kit (Roche) was used to detect hybridisation following the manufacturer's instructions. PCR primers specific for each ST mutant were designed using the sequencing data for each mutant. The primer pair (STM13-KE-C′-1-Rev and STM13-KF-C′) used to confirm the re-isolation of wild-type strain Ap3AS targeted the region identified when sequencing ST mutants 13-1, 13-2 and 13-3 (Table 2).

PCR reactions were conducted using 2 μl of extracted DNA as template in a 20 μl reaction containing 2 μl of 10× reaction buffer, 1 μM of each primer, 200 μM of each dNTP and 1.5 U of Taq DNA polymerase (Promega). PCRs were incubated at 95° C. for 2 min, followed by 35 cycles of 94° C. for 45 s, 52° C. for 45 s and 72° C. for 1 min, with a final incubation at 72° C. for 7 min.

### Histological Examination

### Preparation of Tracheal Sections

Samples of upper, middle and lower trachea were collected and immersed in 10% neutral buffered formalin (10% formalin, 4 g NaH₂PO₄ and 6.5 g Na₂HPO₄ per litre) for at least 24 h for fixation. Tissues were then processed into paraffin wax, followed by vacuum embedding. Sections 2 μm thick were cut and collected onto glass slides. Following dewaxing and rehydration, the sections were stained with haematoxylin and eosin, and examined by light microscopy for lesions and for measurement of mucosal thickness.

### Examination of Tracheal Lesions and Measurement of Mucosal Thickness

Histological lesions in upper, middle and lower sections of trachea were scored for severity on a scale of 0 to 3 as follows:

0=no significant changes;

0.5=very small aggregates of lymphocytes (less than 2 foci) or very slight, diffuse lymphocytic infiltration;

1=small aggregates of lymphocytes (more than 2 foci) or minor thickening of the mucosa caused by diffuse infiltration of lymphocytes;

2=moderate thickening of the mucosa due to heterophil and lymphocyte infiltration, as well as oedema accompanied by degeneration of epithelia with or without luminal exudation;

3=considerable thickening caused by infiltration of heterophils and lymphocytes and oedema with squamous metaplasia or epithelial degeneration and luminal exudation.

The mucosal thickness of the trachea of each bird was determined by measuring the thickness at 6 points on each section from the upper, middle and lower trachea from each bird. The mean thickness in micrometres was then calculated for each of the three regions.

### Statistical Analyses

Median tracheal histological lesion scores for each experimental group were compared using Mann-Whitney U tests (Minitab version 14.2 for Windows). Student's t-test and a one-way analysis of variance (ANOVA) were used to compare the mean tracheal mucosal thicknesses. A probability (P value)≦0.05 was regarded as significant.

### SDS-PAGE Electrophoresis

The cells in a 1 ml sample of a mid-log phase culture of each of the ST mutants, as well as the Ap3AS and ts-11 strains, were collected by centrifugation at 16,000 g for 5 min, then resuspended in 1×SDS-PAGE lysis buffer and incubated at 100° C. for 5 min before rapid cooling on ice. Total cell proteins were separated in a 12.5% polyacrylamide gel together with molecular mass standards (Marker 12™ Wide Range Protein Standard, Novex) and then stained with Coomassie brilliant blue.

### Results

### Virulence and Infectivity of ST Mutants

### Clinical Signs and Post Mortem Examination

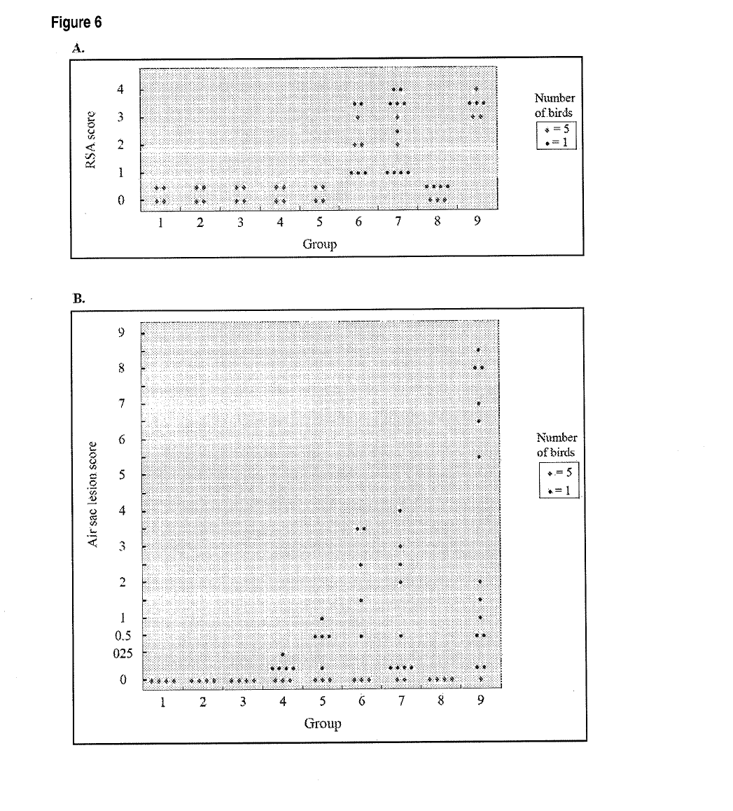

Air sac lesions were not seen in birds exposed to aerosols of ST mutants 04-1 (Group 2), 33-1 (Group 3) or 22-1 (Group 8), or in the negative control birds (Group 1). Mild lesions (score of 0.25) were observed in one bird inoculated with ST mutant 03-1 (Group 4). Of the 20 birds infected with ST mutant 26-1 (Group 5), four had mild lesions (0.50 to 1.00), whilst lesions were only seen in the abdominal air sacs of four birds exposed to ST mutant 18-1 (Group 6). Six of 20 birds had mild to severe lesions (0.50 to 2.50) in the group infected with ST mutant 20-1 (Group 7), while mild to severe lesions (0.50 to 3.00) were seen in 11/18 birds infected with the virulent Ap3AS strain (Group 9). The results are summarised in Table 9 and shown graphically in FIG. 6.

TABLE 9
RSA results, air sac lesion scores and re-isolation rate of M. gallisepticum
from birds in the virulence and infectivity study.

| | | | | MA plate^{#} | | | |
|---|---|---|---|---|---|---|---|
| | In- | RSA* | Air sac | Air | | MB\hat{ } | |
| Group | oculum | Week 2 | lesions* | sacs | Trachea | Air sacs | Trachea |
| 1 | Medium | 0/20 | 0/20 | 0/20 | 0/20 | 0/20 | 0/20 |
| 2 | ST | 0/20 | 0/20 | 0/20 | 0/20 | 0/20 | 0/20 |
| | mutant | | | | | | |
| | 04-1 | | | | | | |
| 3 | ST | 0/20 | 0/20 | 0/20 | 0/20 | 0/20 | 2/20 |
| | mutant | | | | | | |
| | 33-1 | | | | | | |
| 4 | ST | 1/20 | 1/20 | 0/20 | 2/20 | 0/20 | 5/20 |
| | mutant | | | | | | |
| | 03-1 | | | | | | |
| 5 | ST | 0/20 | 4/20 | 1/20 | 6/20 | 1/20 | 10/20 |
| | mutant | | | | | | |
| | 26-1 | | | | | | |
| 6 | ST | 20/20 | 5/20 | 5/20 | 17/20 | 7/20 | 19/20 |
| | mutant | | | | | | |
| | 18-1 | | | | | | |
| 7 | ST | 20/20 | 6/20 | 7/20 | 20/20 | 8/20 | 20/20 |
| | mutant | | | | | | |
| | 20-1 | | | | | | |
| 8 | ST | 0/19 | 0/19 | 0/19 | 1/19 | 0/19 | 3/19 |
| | mutant | | | | | | |
| | 22-1 | | | | | | |
| 9 | Ap3AS | 18/18 | 11/18 | 9/18 | 18/18 | 10/18 | 15/18 |

*Results are indicated as number of positive responses/number examined. Complete RSA and air sac lesion score data are shown in Appendix E. One bird died in Group 8 and two birds died in Group 9 before post mortem
^{#}Results are indicated as number of positive samples/number collected.
\hat{ }Results are indicated as number of colour-change samples/number collected. Signature tags were only detected as the ones carried by ST mutants that infected birds in Groups 2 to 8.

### Antibody Responses and Re-Isolation of ST Mutants

No anti-mycoplasma antibody was detected at the time of infection in the serum of any experimental birds using the RSA test. Two weeks after infection, antibody responses were not detected in any of the birds in Groups 2, 3, 5 and 8, whilst a response was detectable in only one bird in Group 4. In contrast, strong RSA reactions against M. gallisepticum were detectable in all the birds in Groups 6 and 7. The negative control birds (Group 1) did not show reactivity against Mg in the RSA test, is while all the positive control birds (Group 9) had strong RSA reactions against Mg (Table 9, FIG. 6). M. gallisepticum were not isolated on MA plates inoculated with swabs of the air sacs or trachea of any bird in Group 2 (ST mutant 04-1 infected) or Group 3 (ST mutant 33-1 infected) (Table 9). M. gallisepticum was not isolated on MA plates inoculated with swabs of the air sacs of any birds in Groups 4 (ST mutant 03-1 infected) or 8 (ST mutant 22-1 infected), but they were isolated from the trachea of two birds in Group 4 and one bird in Group 8. In Group 5 (ST mutant 26-1 infected), M. gallisepticum were isolated from the air sacs of one bird and the tracheas of 6 birds. M. gallisepticum were also isolated from the tracheas of 17/20 birds in Group 6 (ST mutant 18-1 infected) and all the birds in Group 7 (ST mutant 20-1 infected), and also from swabs of the air sacs of 5/20 birds in Group 6 and 7/20 birds in Group 7. In the positive control group (Group 9), M. gallisepticum were isolated from the air sacs of 9/18 birds and the tracheas of 17/18 birds. M. gallisepticum were more frequently isolated from swabs incubated in MB than on MA plates. M. gallisepticum was not isolated in MB inoculated with swabs of the air sacs or tracheas of any bird exposed to ST mutant 04-1 (Group 2) (Table 9). In the other mutant-infected groups, Mg was recovered in MB inoculated with swabs of the air sacs of one bird in Group 5 (ST mutant 26-1), 7 birds in Group 6 (ST mutant 18-1) and 8 birds in Group 7 (ST mutant 20-1). Mg were recovered from the tracheas of birds in 6 of the groups exposed to ST mutants, with the number of infected birds ranging from 2 in Group 3 (ST mutant 33-1 infected) to 20 in Group 7 (ST mutant 20-1 infected). The identity of the recovered M. gallisepticum ST mutants was confirmed using the unique signature tags they carried (Table 9).

### Tracheal Lesions and Mucosal Thicknesses

Median tracheal lesion scores are shown in Table 10. The scores of birds in all the mutant infected groups differed significantly from those of birds in the positive control group (Group 9) (P<0.0001). There was no significant difference between the negative control group (Group 1) and Groups 5 (ST mutant 26-1 infected) or 7 (ST mutant 20-1 infected). The lower tracheal lesion scores of the birds infected with ST mutant 18-1 (Group 6) did not differ from those of birds in Groups 2, 3, 4, 5 and 7, but did differ significantly from those of birds in Group 8 (ST mutant 22-1 infected) and in is the negative control group (Group 1), while only lesion scores in the middle trachea of the birds in the positive control group differed from those of birds in any of the other groups. Mean tracheal mucosal thicknesses are shown in Table 10. The mean mucosal thicknesses in the middle trachea did not differ significantly between any of the mutant infected groups (Groups 2 to 8). However, they were all significantly greater than that of the negative control group (Group 1), except for those in Groups 2 and 7, and they were significantly less than that of the positive control group (Group 9). There was no significant difference between the negative controls and Group 7 (ST mutant 20-1 infected) in the mucosal thicknesses of the upper and middle tracheas, but there was a difference in the lower trachea (P=0.004).

In summary, the positive controls had significantly more severe tracheal lesions, as assessed by histological lesion score or mucosal thickness, than any of the groups infected with the ST mutants. Group 4 (ST mutant 03-1 infected) was the most severely affected among the groups infected with the ST mutants.

TABLE 10
Tracheal lesion scores and mucosal thicknesses in birds in the virulence and infectivity study.

| | | Median tracheal lesion | | | Mean tracheal mucosal | | |
|---|---|---|---|---|---|---|---|
| | | score (minimum, maximum) | | | thickness ± SD* (μm) | | |
| Group | Inoculum | Upper | Middle | Lower | Upper | Middle | Lower |
| 1 | Medium (−) | 0.25 (0, 0.5)^{a} | 0.25 (0, 0.5)^{a} | 0 (0, 0.25)^{a} | 50 ± 11^{a} | 41 ± 8^{a} | 37 ± 6^{a} |
| 2 | ST mutant 04-1 | 0.5 (0.25, 1.5)^{b} | 0.25 (0, 0.5)^{b,c} | 0.25 (0, 0.25)^{a,b} | 67 ± 11^{b} | 53 ± 12^{a.b} | 40 ± 9^{a,b,c} |
| 3 | ST mutant 33-1 | 0.75 (0.25, 3)^{b} | 0.25 (0, 2.5)^{a,b} | 0 (0, 0.25)^{a,b} | 73 ± 32^{b,c} | 47 ± 13^{b} | 38 ± 8^{b} |
| 4 | ST mutant 03-1 | 1 (0.25, 3)^{b} | 0.375 (0, 1.5)^{c} | 0.25 (0, 1.5)^{c} | 89 ± 45^{d} | 54 ± 12^{b} | 46 ± 22^{c} |
| 5 | ST mutant 26-1 | 0.5 (0, 2)^{a,b} | 0.25 (0, 1)^{a,b} | 0 (0, 2)^{a,c} | 64 ± 24^{b,e} | 49 ± 14^{b} | 48 ± 22^{b,c} |
| 6 | ST mutant 18-1 | 0.25 (0, 10)^{a} | 0.25 (0, 1.5)^{a,b,c} | 0.25 (0, 1.5)^{b,c} | 62 ± 15^{b,e} | 53 ±15^{b} | 49 ± 15^{c,d} |
| 7 | ST mutant 20-1 | 0.25 (0, 1.5)^{a} | 0.125 (0, 1.5)^{a} | 0.125 (0, 1.0)^{a,c} | 56 ± 22^{a,c,e} | 49 ± 23^{a,b} | 51 ± 19^{c,d} |
| 8 | ST mutant 22-1 | 0.5 (0, 1)^{a,b} | 0.25 (0, 1)^{a,b} | 0 (0, 0.25)^{a} | 64 ± 13^{b,e} | 49 ± 8^{b} | 39 ± 8^{a,b} |
| 9 | Ap3AS (+) | 1.5 (1, 3)^{c} | 1.50 (0.5, 3)^{d} | 1.50 (0.25, 3)^{d} | 168 ± 70^{f} | 153 ± 84^{c} | 120 ± 56^{e} |

*SD: standard deviation
Data in the same column with the same superscript are not significantly different.
Complete data are shown in Appendix F.

### Example 4

### Evaluation of Protection Provided by Vaccination with a Selected M. gallisepticum ST Mutant

### Preparation of ST Mutant Vaccine

ST mutant 26-1 was grown in MB supplemented with 160 μg gentamicin/ml at 37° C. The method used to determine the concentration of organisms in the culture was described above. The concentration of ST mutant 26-1 was adjusted to approximately 1×10⁷ CCU/drop (22.5 μl) for vaccination and each bird was then vaccinated intra-ocularly with a drop of the culture.

### Experimental Design

A total of eighty 5-week-old SPF chickens were randomly assigned into 4 groups and housed in positive pressure fibreglass isolators. ST mutant 26-1 was administered by eye-drop at day 0. Wild-type Ap3AS was administered by aerosol to challenge the birds 2 weeks after immunisation. The birds in Group 1 served as the negative controls. These birds were vaccinated with MB by eye-drop at day 0 and challenged with MB by aerosol at day 14. The birds in Group 2 served as the positive controls. They were vaccinated with MB by eye-drop at day 0 and infected with wild-type Ap3AS by aerosol at day 14. The birds in Group 3 served as the vaccination controls. These birds were administered ST mutant 26-1 by eye-drop at day 0. MB was then given by aerosol 14 days after vaccination. The birds in Group 4 served as the vaccinated and challenged group. Chickens were immunised with ST mutant 26-1 by eye-drop at day 0 and challenged with wild-type Ap3AS by aerosol at day 14.

Sample Collection from Experimental Chickens

Blood samples were collected from all chickens prior to vaccination, challenge and euthanasia. Sera were then harvested and tested by RSA to determine concentrations of anti-mycoplasma antibody levels. The body weight of each bird was also measured before immunisation, at challenge and at post mortem. At day 28 (14 days after challenge), chickens were euthanased and subjected to post mortem examination, and samples taken for culture of M. gallisepticum. The six air sacs were visually assessed for lesions and scored from 0 to 3 as described above. Swabs were taken from the air sacs and tracheas of birds and streaked onto MA without gentamicin added and then transferred into MB containing no gentamicin to isolate M. gallisepticum. The MA plates were incubated at 37° C. for 10 days and the MB cultures were incubated at 37° C. until a colour change was observed. Organisms from broths showing a colour change were pelleted by centrifugation and subjected to PCR amplification to identify ST mutant 26-1 and wild-type Ap3AS (above). Samples of the upper, middle and lower regions of the trachea of each bird were collected and fixed in 10% neutral buffered formalin for at least 24 h. Following processing and staining, sections were examined by light microscopy. Lesions were scored for severity on a scale of 0 to 3, as described above. The mucosal thickness was measured for each tracheal region at 6 different points and the mean thickness was then calculated for each of the three regions.

Re-Isolation of M. gallisepticum

A pair of PCR primers that was specific for ST mutant 26-1 (IGstmGenmeF3 and STM26-Rev) and the P2/P4 primer set, which amplified the unique tag region, was used to confirm the re-isolation of ST mutant 26-1. The primer pair S™26-genome and STM26-Rev bound either side of the transposon insertion site in ST mutant 26-1 and was used to confirm the re-isolation of wild-type Ap3AS. This PCR could easily differentiate the ST mutant 26-1 from the Ap3AS parent strain (Table 2). PCRs were is conducted with 2 μl extracted DNA as template in a 20 μl reaction containing 2 μl of 10× reaction buffer, 1 μM each primer, 200 μM of each dNTP and 1.5 U of Taq DNA polymerase (Promega). PCRs were incubated at 95° C. for 2 min, then through 35 cycles of 94° C. for 45 s, 52° C. for 45 s and 72° C. for 1 min, with a final incubation at 72° C. for 7 min. The resultant products were separated in a 2% agarose gel together with DNA molecular weight markers.

### Statistical Analyses

The mean percentage body weight gains for each group were analysed for differences using a one-way ANOVA and Student's t-test (Minitabs v14.2 for Windows). Median tracheal lesion scores were compared using Mann-Whitney U tests. Mean mucosal thicknesses in the upper, middle and lower trachea were compared using Student's t-test and a one-way ANOVA. A P value≦0.05 was regarded as significant.

### Results

### Gross Lesions

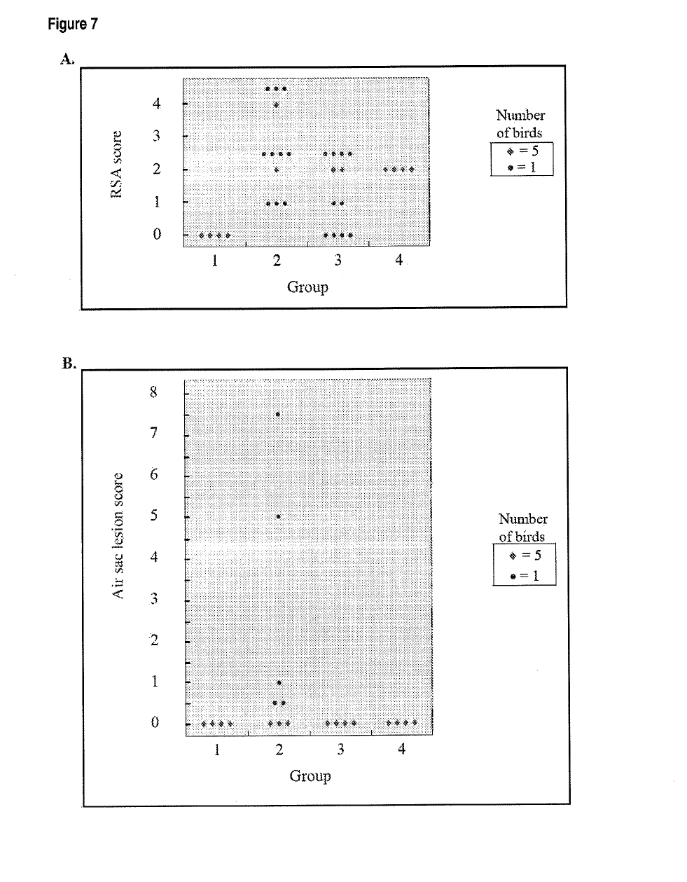

Gross air sac lesions were only seen in birds in the positive control group (Group 2). In this group, 5 birds had air sac lesions, with total air sac lesion scores ranging from 0.5 to 2.5. The results are summarised in Table 10 and shown graphically in FIG. 7.

TABLE 10
Results of RSA test, air sac lesion scores and re-isolation of M. gallisepticum of all groups in
the immune protection study.

| | | | | | | | | MB^{&} | | | |
|---|---|---|---|---|---|---|---|---|---|---|---|
| | | | RSA^{#} | | Air sac | MA plate\hat{ } | | Air sacs | | Trachea | |
| Group | Vaccination | Challenge | Day 14 | Day 28 | lesions | Air sacs | Trachea | Mutant 26-1 | Ap3AS | Mutant 26-1 | Ap3AS |
| 1 | Medium | Medium | 0/20 | 0/20 | 0/20 | 0/20 | 0/20 | 0/20 | 0/20 | 0/20 | 0/20 |
| 2 | Medium | Wt Ap3AS* | 0/20 | 20/20 | 5/20 | 1/20 | 19/20 | 0/20 | 2/20 | 0/20 | 10/20 |
| 3 | ST mutant 26-1 | Medium | 0/20 | 16/20 | 0/20 | 1/20 | 2/20 | 1/20 | 0/20 | 2/20 | 0/20 |
| 4 | ST mutant 26-1 | Wt Ap3AS | 0/20 | 20/20 | 0/20 | 0/20 | 7/20 | 0/20 | 0/20 | 0/20 | 9/20 |

*wild-type Ap3AS
^{#}Complete RSA and air sac lesion score data are shown in Appendix G.
\hat{ }Number of samples showing M. gallisepticum colonies growing on the MA plate/total number of samples collected.
^{&}PCRs were conducted to determine the presence of ST mutant 26-1 and strain Ap3AS.

Serological Testing and Re-Isolation of M. gallisepticum

Sera collected from birds at the time of vaccination and challenge contained no detectable anti-mycoplasma antibody. None of the chickens in the negative control group (Group 1) had any detectable serum antibody against M. gallisepticum at the time of post mortem. In contrast, a large proportion of birds in the other 3 groups had detectable antibodies against M. gallisepticum at post mortem. Every bird in the positive control group (Group 2) had produced a moderate to strong RSA antibody response against M. gallisepticum. Anti-mycoplasma antibodies were detected in 16/20 serum samples from Group 3, with the RSA scores ranging from low to moderate. Moderate anti-mycoplasma antibody titres were detected in all birds in Group 4 (vaccinated with ST mutant 26-1 and infected with the wild-type strain). The results are summarised in Table 10. M. gallisepticum were more frequently isolated from tracheal swabs than swabs of the air sacs in Groups 2, 3 and 4, and were not isolated from the air sacs or tracheas of any bird in Group 1 (negative controls). Mg colonies is were grown from swabs of the air sacs in one bird each in Groups 2 (challenged only) and 3 (vaccinated only), whilst re-isolation was achieved from the trachea of 19 and 2 birds, respectively. In Group 4, which had been given ST mutant 26-1 as a vaccine and then infected with strain Ap3AS, Mg was recovered on MA plates from the air sacs of one bird and from the tracheas of 7 birds (Table 10). As in previous experiments, re-isolation rates were higher in MB than on MA plates. PCR amplification confirmed that the only mycoplasmas re-isolated from Groups 2 and 3 were the strain that had been administered to that group. Following PCR amplification using strain specific primer pairs, the only mycoplasmas re-isolated from Group 4 were wild-type Ap3AS. The results are shown in Table 10.

### Weight Gains of Chickens

There was no significant difference in weight gain between any of the groups over the 14 days following vaccination. At week 4 (14 days after challenge), the positive control birds (Group 2) had lower mean weight gains than the birds in Groups 1 (negative controls) or 3 (vaccination controls) (P=0.006 and 0.027, respectively), but not than the birds in Group 4 (vaccinated and challenged) (P=0.332) (Table 11). In the period between challenge and post mortem (day 14 to day 28), the mean weight gain of the chickens in Group 2 (challenged only) was significantly lower than that of the birds in each of the other groups. The mean weight gain of birds in Group 3 (vaccinated, not challenged) was not greater than that of the negative controls, but was significantly greater than that of the birds in Group 4 (vaccinated and challenged) (P=0.016). However, the mean weight gain of the birds in Group 4 did not differ significantly from that of the birds in Group 1 (P=0.580). The results are shown in Table 11.

### Tracheal Lesions and Mucosal Thicknesses

The median tracheal lesion scores are shown in Table 12. The lesions were severe in the birds in Group 2 (challenged only). The median tracheal lesion scores of birds in Group 2 (positive controls) differed significantly from those in the other three groups (P<0.0001). The lesion scores of the birds in the other groups did not differ significantly from each other. The mean mucosal thicknesses of the upper, middle and lower trachea of the birds in Group 2 (challenged only) were significantly greater than those of the birds in the other three groups (P<0.0001), but there was no significant is difference between those of the birds in these other three groups (Table 12). However, in middle and lower tracheas, mean mucosal thickness was less in the birds in Group 4 than in the birds in Groups 1 (P=0.028) or 3 (P=0.036).

TABLE 11
Weight gain of chickens in all experimental groups in the immune protection study

| | | | Day 0 | Day 0-14 | Day 0-28 | Day 14-28 |
|---|---|---|---|---|---|---|
| Group | Vaccination | Challenge | weight (g) | gain (%) | gain (%) | gain (%) |
| 1 | Medium | Medium | 382 ± 41 | 53 ± 6^{a} | 106 ± 12^{a} | 34 ± 7^{a,c,d} |
| 2 | Medium | Wt Ap3AS | 422 ± 33 | 52 ± 7^{a} | 94 ± 13^{b} | 28 ± 4^{b} |
| 3 | ST mutant 26-1 | Medium | 387 ± 43 | 48 ± 10^{a} | 104 ± 15^{a} | 38 ± 6^{c} |
| 4 | ST mutant 26-1 | Wt Ap3AS | 415 ± 58 | 50 ± 9^{a} | 100 ± 13^{a,b} | 33 ± 6^{d} |

Results are expressed as mean ± standard deviation.
Values with the same superscript in the same column are not significantly different.
Complete data are shown in Appendix H.

TABLE 12
Tracheal lesion scores and mucosal thicknesses of birds in the immune protection study

| | | | Median tracheal | | | Tracheal mucosal | | |
|---|---|---|---|---|---|---|---|---|
| | | | lesions (minimum, maximum) | | | thickness ± SD^{#} (μm) | | |
| Group | Vaccination | Challenge | Upper | Middle | Lower | Upper | Middle | Lower |
| 1 | Medium | Medium | 0 (0, 0.5)^{a} | 0 (0, 0)^{a} | 0 (0, 0)^{a} | 41 ± 6^{a} | 32 ± 5^{a} | 28 ± 4^{a} |
| 2 | Medium | Wt Ap3AS* | 2.5 (0.5, 3)^{b} | 2.5 (0.5, 3)^{b} | 2 (1, 3)^{b} | 233 ± 101^{b} | 206 ± 107^{b} | 173 ± 98^{b} |
| 3 | ST mutant 26-1 | Medium | 0 (0, 0.25)^{a} | 0 (0, 0.5)^{a} | 0 (0, 0.25)^{a} | 43 ± 11^{a} | 34 ± 8^{a} | 28 ± 6^{a} |
| 4 | ST mutant 26-1 | Wt Ap3AS | 0 (0, 1)^{a} | 0 (0, 0.25)^{a} | 0 (0, 0)^{a} | 44 ± 14^{a} | 28 ± 5^{c} | 25 ± 3^{c} |

*wild-type strain Ap3AS
^{#}SD: standard deviation
Data in the same column with the same superscripts are not significantly different.
Complete data are shown in Appendix I.

### Example 5

### Knockout Mutants of the dppD and oppD Oligopeptide Transporter Genes in the Avian Pathogenic Escherichia coli Strain E956 (APEC E956)

In vivo safety studies showed statistically significant attenuation of the oppD knockout both in terms of lesion scores and re-isolation rates from birds, as compared to the dppD knockout or the parent E956. In vivo efficacy studies showed statistically significant protection of oppD knockout vaccinated birds following challenge with APEC E956.

Examples 1 and 2 illustrate a number of attenuating gene mutations including the dppD and oppD genes, which is homologous to other bacterial genes involved in the transport of oligopeptides into the cell.

As all bacterial species possess near identical transport systems, new vaccines against other avian pathogens, as well as pathogens of other animal species, could be developed by producing dppD knockouts.

This example illustrates knockouts of the dppD and oppD genes in the Avian Pathogenic Escherichia coli strain E956 (APEC E956). Each mutant was tested for safety and efficacy using a challenge system.

### Methodology

E. coli Strains, Media and Plasmids

The avian pathogenic Escherichia coli strain E956 (APEC E956) was originally isolated from a day-old chick at a broiler breeder farm) and was found to be sensitive to the antibiotics ampicillin, sulphafurazole, trimethoprim, chloramphenicol, tetracycline, and kanamycin. The organism was propagated in Luria-Bertani broth (LB) or on LB agar at 30° C. or 37° C. overnight with the appropriate antibiotic selection (ampicillin, 50 ug/ml; kanamycin, 100 ug/ml) unless otherwise stated. The E. coli DH5α strain was used for standard cloning and plasmid production.

The red recombinase system was used to promote homologous recombination between the knockout constructs and the gene knockout target using the temperature sensitive pKD46 plasmid carrying an ampicillin resistance gene, the three “Red system” genes γ, λ, and exo coding for Gam, Bet, and Exo respectively. The knockout constructs were assembled by PCR and ligated to the pGEM-T vector (Promega). The plasmid containing each construct was used as template in PCR to produce linear DNA for transformation.

### Preparation of Gene Knockout Constructs

The kanamycin gene was amplified from the transposon TnphoA using the oligonucleotide primers TXkanFor and TYkanRev (Table 13). The resultant 1.064 kbp PCR product was purified using the UltraClean PCR purification kit (MoBio, California USA) following the manufacturer's instructions and ligated to pGEM-T vector (Promega) following the manufacturer's protocols. The ligation mixture was used to transform E. coli DH5α by electroporation using a Gene Pulser (BioRad) with the settings 2,500 V, 200Ω, 250 μF. and recombinants selected on LB agar containing 25 μg/ml of kanamycin. Transformants were grown overnight in LB broth containing 25 μg/ml of kanamycin and plasmid extracted using kit (Promega) following the manufacturer's instructions. Cloning of the kanamycin resistance gene was verified by restriction endonuclease analysis. Linear DNA was used for transformation: for dppD knockouts the DNA was prepared by PCR using the primer pair WE and WF each containing 40 by 5′ regions complementary to regions 4384-4423 by and 4829-4868 by respectively within the dppD gene of the GenBank Accession L08399. The WE and WF oligonucleotide primers contained 20 by 3′ regions complementary to the kanamycin gene and when used in PCR amplified the kanamycin gene with 40 by ends complementary to the dppD DNA.

TABLE 13
Oligonucleotides used in this study

| | | SEQ | |
|---|---|---|---|
| Oligonucleotides | Sequence (5′-3′) | ID No. | PCR product |
| TXkanFor | tagactgggcggttttatgg | 73 | Kanamycin |
| TYkanRev | cgaagcccaacctttcatag | 74 | Kanamycin |
| TZ5′dppD | cgtagaccgcatcagctaca | 75 | dppD |
| UA3′dppD | gtgggtaatggcatttggac | 76 | dppD |
| WAKanFshort | cgttggctacccgtgatatt | 77 | Kanamycin |
| WE_dppD5′_kanF | ccgtcagcgagcgattgacctgctga | 78 | dppD/Kan 5′ |
| | atcaggtcggtatttagactgggcgg | | |
| | ttttatgg | | |
| WF_dppD3′_kanR | gcgggttaagcaggcagccgttcggg | 79 | dppD/Kan 3′ |
| | cggtcgtacttgcccgaagcccaacc | | |
| | tttcatag | | |
| WSoppDFor | gcactgctgaacgtgaaaga | 80 | oppD |
| WToppDRev | ctccaccggtttaaagcaag | 81 | oppD |
| WU1oppDRev + Kan | ccataaaaccgcccagtctattttta | 82 | oppD arm 5′ |
| | ccgcatcgagcatc | | |
| WV2oppDFor + Kan | ctatgaaaggttgggcttcgtcgacc | 83 | oppD arm 3′ |
| | taagctgctgattg | | |
| Sidfwdseq | ctttccaccctgcacctaag | 84 | iucA |
| bwd siderophore | ctcacgggtgaaaatatttt | 85 | iucA |
| 16Sfwd | gctgacgagtggcggacggg | 86 | 16s RNA |
| 16Srev | taggagtctggaccgtgtct | 87 | 16s RNA |

### Production of Gene Knockouts

The APEC strains E956 was transformed with pKD46 by electroporation using the Bio-Rad Gene Pulser (Bio-Rad) set at 2,500 V, 200Ω, 250 μF. Recombinants were selected on Luria-Bertani agar containing 100 μg/ml of ampicillin following incubation at 30° C. overnight. We produced gene knockouts through homologous recombination using known methods. A single fresh colony of APEC E956/pKD46 was placed into 5 ml LB containing 100 μg/ml ampicillin and incubated with shaking (200 rpm) at 30° C. for 16 hours. The following day, a 1:50 dilution of an overnight culture was made in a volume of 20 ml of LB containing 100 μg/ml ampicillin and the cultures shaken (200 rpm) at 30° C. in a 125 ml conical flask. At a concentration of 10⁷ cells/ml, L-arabinose (Sigma) was added to a final concentration of 1 mM. Cultures were induced for approximately 1 hour at 30° C. and the cells then heat shocked for 15 minutes at 42° C. and immediately transferred to an ice-water bath for 10 mins, then collected by centrifugation at 10,000×g for 10 mins at 4° C. The cells were resuspended in 1 ml of ice-cold 1 mM MOPS containing 20% glycerol, transferred to an 1.5 ml centrifuge tube and centrifuged at 16,000×g for 30 s at 4° C. in a bench-top microfuge. The supernatant was removed and cells resuspended in 1 ml of 1 mM MOPS containing 20% glycerol and centrifuged as before. This step was repeated once more and the cells were finally resuspended in 100 μl 1 mM MOPS containing 20% glycerol. The cells were then added to pre-cooled electroporation cuvettes (2 mm gap, Bio-Rad) and 50 μl of cells with 300 ng of DpnI digested DNA was transformed using the previous settings. Immediately following electroporation, the cells were recovered by suspension in 0.3 ml of LB, diluted into 2.7 ml LB and incubated at 37° C. with shaking for 1.5 h. The culture was 5× concentrated in LB and 0.2 ml inoculated onto LB plates containing 20 μg/ml kanamycin and incubated overnight at 37° C. Transformants were selected and grown in LB containing 40 ug/ml kanamycin and gene knockouts confirmed by PCR and Southern blotting.

Confirmation of dppD and oppD Gene Knockout Strains

To verify the insertion of the kanamycin resistance gene in dppD or oppD, PCR was conducted using the primer pair for dppD (TZIUA) and oppD (WSIWT) to amplify the respective gene region. The predicted size of the PCR product for APEC E956 parent for dppD and oppD would be 0.89 kbp and 0.951 kbp respectively. The predicted size increase with insertion of the kanamycin gene (1.064 kbp) would be 1.774 kbp and 1.924 kbp for dppD and oppD respectively.

### Genomic DNA Extraction and Southern Blot

Genomic DNA of APEC E956, ΔdppD, and ΔoppD strains was prepared using phenol/chloroform as previously described (Sambrook et al., 2001). Genomic DNA from E. coli strains was digested with PstI, and the fragments together with molecular weight markers were separated by agarose gel electrophoresis. Following agarose gel electrophoresis genomic DNA fragments was transferred from the gel to a nylon membrane (Hybond-N⁺, GE Healthcare) by capillary transfer. DNA probes were labeled with [γ³²P]dATP using a random-primed DNA-labeling kit (Roche). Prehybridization and hybridization were carried out in Church buffer (0.5 M Na₂HP0₄ [pH 7.4], 7% sodium-dodecyl sulfate, 1 mM EDTA, 1% bovine serum albumin BSA) (Church & Gilbert, 1984) overnight at 54° C. Membranes were washed in 2×SSC (1×SSC is 0.15 M NaCl plus 0.015M sodium citrate}-0.1% sodium dodecyl sulfate twice at 54° C. for 5 min each and then once in 0.5×SSC, 0.1% SDS for 15 min at 65° C. and autoradiographed with Kodak BioMax MS film at −70° C.

Safety of APEC E956 dppD and oppD Knockouts

One-day-old chicks were infected with APEC E956, ΔdppD, and ΔoppD strains to assess the pathogenicity of each mutant compared to uninfected and APEC E956 infected. A total of 95 one-day-old chicks were purchased (SPAFAS, Woodend Vic) and allocated to three groups of 20 chicks each and one group of 35. The birds were housed in positive pressure isolators and fed ad libitum on irradiated commercial starter feed. Cloacal swabs were taken from two chicks each from each group and streaked onto MacConkey agar to assess commensal E. coli. Overnight cultures of E956 ΔdppD, ΔoppD in nutrient broth with 40 ug/ml kanamycin together with E956 without kanamycin were prepared to give a concentration of 1E+10 colony forming units/ml (cfu/ml), All groups were inoculated by eye-drop with 10 times the normal immunising dose of the commercial infectious bronchitis virus vaccine Vic S (Websters, Australia). Groups 2 and 3 of 20 chicks each received 20 ml of an aerosol containing 1E+10 cfu/ml of ΔdppD and ΔoppD) respectively whilst group 4 of 35 birds received 20 ml of 1E+10 cfu/ml of E956. Chicks in Group 1 were left untreated and served as the negative control. The chicks from Groups 2, 3 and 4 were exposed a further two times to the same APEC strain at 3 and 5 days of age. All birds were subjected to post mortem at 10 days of age. Disease was assessed by gross pathology, air sac lesions were scored according to previous criteria. Swabs were taken from the left and right posterior and anterior airsacs, the trachea and aseptically from the liver. The swabs were inoculated onto MacConkey agar with and without kanamycin (40 ug/ml) and incubated overnight at 37° C. The next day the plates were observed for the presence of “brick-red” colonies (typical E. coli phenotype) their numbers were counted and recorded. If the number of colonies was less than 30 then PCR was conducted to identify E. coli strain. The APEC strains were identified using the primer pairs: for dppD WA/UA, for oppD WA/WT, for E956 Sidfwdseq/bwd siderophore (Table 13) which amplifies the iucA gene present on pVM01 and for commensal E. coli the 16s rDNA was amplified with 16Sfwd/16Srev.

Efficacy of APEC dppD and oppD Knockouts

To assess the efficacy of the E. coli vaccine candidate, sixty one-day-old birds were divided into three groups of 20 birds each. Groups 2 and 3 were aerosol vaccinated with E956 ΔdppD, ΔoppD at day one as above whilst Group 1 remained unvaccinated as a control and housed separately in isolators as above. On day 12 post-vaccination all groups were separately challenged with 20 ml of an aerosol containing 1E+10 cfu/ml of APEC E956 as above and replaced in their isolators. After 4 days all birds were euthanased by inhalation of halothane and subjected to post mortem. Airsac lesion scores were assessed as described above and swabs taken for re-isolation of infecting organisms were cultured on MacConkey agar with and without kanamycin (40 ug/ml).

### Analysis of Results

Statistical analysis was performed on the rate and degree of airsac lesion scores, isolation rate of organisms and weight gain of birds. The tests used were the Fisher's Exact Test for reisolation rates and the Mann-Whitney U Test for lesion scores and weight change.

### Results

Development and Verification of APEC E956 ΔdppD, and ΔoppD

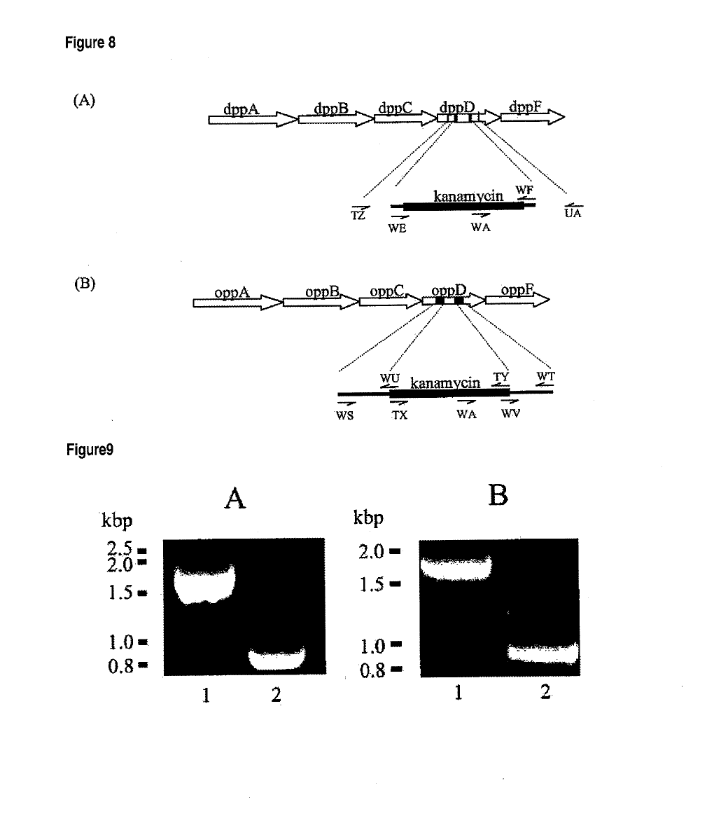

Linear DNA constructs for specific gene knockouts produced by PCR were used to transform APEC E956/pKD46. Transformants were selected on LB agar containing 40 μg/ml of kanamycin and then PCR conducted to confirm clones were carrying the kanamycin gene. The size of the amplicon from E956 ΔdppD using the oligonucleotide primer pair TZ/UA was predicted to be 1.774 kbp due to the insertion of the kanamycin DNA (1.064 kbp) as compared to the E956 parental strain (0.89 kbp) FIG. 8A and FIG. 9, Panel A, lanes 1 and 2 respectively. Similarly, the size of the amplicon for E956 ΔoppD using the oligonucleotide primer pair WS/WT was close to the predicted size of 1.924 kbp with the E956 parent strain producing an amplicon of 0.951 kbp, FIG. 8B and FIG. 9, Panel B, lanes 1 and 2 respectively.

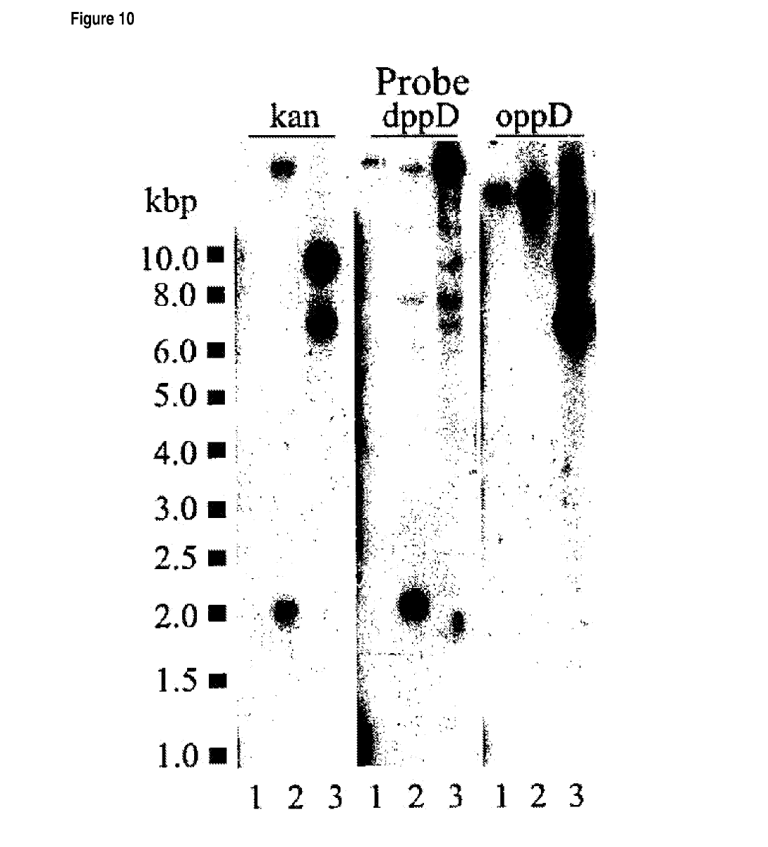

Genomic DNA of APEC strains E956, ΔdppD and ΔoppD were digested with the PstI restriction enzyme, this enzyme was chosen as the kanamycin resistance gene is has a single PstI site in the middle of the gene. The dppD and oppD radiolabeled probes detected bands in APEC E956, ΔdppD and ΔoppD strains (FIG. 10). The dppD probe bound to a similar sized fragment in E956 and ΔoppD strains (FIG. 10, lanes 1 and 3) but to a slightly lower molecular weight band and another band of 2 kbp. The oppD probe bound to a similar sized fragment in E956 and ΔdppD strains (FIG. 10, lanes 1 and 2) and to bands of 9.8 and 7 kbp in ΔdppD (FIG. 10, lane 3). The kanamycin gene probe did not bind to E956 but to the same bands bound by the dppD and oppD probes in ΔdppD and ΔoppD strains (FIG. 10, lanes 1 and 2).

Virulence Studies Using E956 ΔdppD and ΔoppD Strains
The chickens were examined at post mortem for signs of colibacillosis, the six airsacs were examined for lesions and a sample swab taken from the left and right anterior airsac, the trachea and liver of each bird for the re-isolation of vaccine, parental or commensal organisms. The swabs were plated onto MacConkey agar with or without kanamycin added.

The results from these studies are discussed below, summarised in Tables 14, 16 and 19 with statistical comparisons made between each of the groups summarised in Tables 15, 17, 18, 20 and 21.

TABLE 14
Weight gain of chickens in pathogenicity and efficacy experiments

| | % Weight gain (g) | | |
|---|---|---|---|
| Challenge | Pathog'ty Expt | Efficacy Expt | |
| (No. birds) | PM-vaccination | Challenge-vacc'n | PM-challenge |
| None (20)\hat{ } | 174.5 ± 30.6^{a} | — | — |
| E956 (23)\hat{ } | 143.0 ± 40.5^{b} | — | — |
| Challenge only | — | 269 ± 29.6^{a} | 17.9 ± 3.7^{a} |
| (20)* | | | |
| ΔdppD (19)\hat{ }, | 143.0 ± 40.5^{b} | 255 ± 54.0^{a} | 19.3 ± 3.1^{a} |
| (19)* | | | |
| ΔoppD (20)\hat{ }, | 155.0 ± 31.1^{ab} | 255 ± 49.0^{a} | 18.4 ± 2.4^{a} |
| (20)* | | | |

\hat{ }Pathogenicity experiment.
*Efficacy experiment.
Values with the same subscript symbols in the same column are not significantly different (P ≧ 0.05, Student's T-Test 2-tailed)

TABLE 15
Probability (P) values for statistical
analysis of weight gains from table 14.

| | Pathogenicity Experiment | | |
|---|---|---|---|
| | ΔdppD | ΔoppD | E956 |
| Non-vacc | 0.006 | 0.053 | 0.007 |
| ΔdppD | | 0.237 | 0.995 |
| ΔoppD | | | 0.291 |

Efficacy Experiment

| | ΔdppD | ΔoppD |
|---|---|---|
| Non-vacc | 0.302 | 0.257 |
| ΔdppD | | 0.990 |

TABLE 16
Safety of oppD and dppD knockouts compared with E. coli E956 strain

| Challenge | Re-isolation rate | | | | Median no. isolated (Log₁₀ + 1) | | Airsac | Median Lesion |
|---|---|---|---|---|---|---|---|---|
| strain | No antibiotics | | | | no antibiotics (range) | | Lesion rate | Score |
| (No. birds) | LPTas | RPTas | Trachea | Liver | Left + Right Airsac | Trachea | (>0.5) | (range) |
| None (20) | 2/20 | 3/20 | 11/20 | 4/20 | 0.00 (0-0.48)^{a} | 0.00 (0-3.70)^{a} | 0/20^{a} | 0.0 (0-0.0)^{a} |
| E956 (23) | 11/23 | 11/23 | 21/23 | 3/23 | 0.48 (0-4.00)^{c} | 2.48 (0-3.70)^{c} | 17/23^{b} | 3.0 (0-20)^{b} |
| ΔdppD (19) | 10/19 | 11/19 | 16/19 | 8/19 | 2.08 (0-4.00)^{b c} | 1.93 (0-3.70)^{b} | 11/19^{bc} | 1.0 (0-16)^{bc} |
| ΔoppD (20) | 9/20 | 7/20 | 10/20 | 4/20 | 0.00 (0-4.00)^{a b} | 0.85 (0-3.70)^{a} | 9/20^{c} | 0.0 (0-20)^{c} |

Values with the same subscript symbols in the same column are not significantly different (P ≧ 0.05 by Mann-Whitney test, Fischer's exact 2-tailed)

TABLE 17
Probability (P) values for statistical analysis of airsac and tracheal
re-isolation rates from table 16.

| | Airsac | | | Trachea | | |
|---|---|---|---|---|---|---|
| Strain | E956 | ΔdppD | ΔoppD | E956 | ΔdppD | ΔoppD |
| Non-vacc | 0.017 | 0.0105 | 0.0399 | 7e−06 | 0.0005 | 0.3589 |
| E956 | | 0.4299 | 0.1696 | | 0.0238 | 0.00012 |
| ΔdppD | | | 0.1231 | | | 0.0089 |

Mann-Whitney test

TABLE 18
Probability (P) values for statistical analysis of airsac lesion score and
rate from table 16.

| | Lesion Score* | | | Lesion Rate\hat{ } | | |
|---|---|---|---|---|---|---|
| Strain | E956 | ΔdppD | ΔoppD | E956 | ΔdppD | ΔoppD |
| Non-vacc | 1e−05 | 0.0003 | 0.0154 | 0.008 | 0.00001 | 0.000003 |
| E956 | | 0.092 | 0.0065 | | 0.0561 | 0.112 |
| ΔdppD | | — | 0.1415 | | | 1.0 |

*Mann-Whitney test,
\hat{ }Fischer's exact 2-tailed

TABLE 19
Efficacy studies using E. coli E956 strain, oppD and dppD knockouts

| | Re-isolation rate | | | | Median no. isolated (Log₁₀ + 1) | | Airsac | Median |
|---|---|---|---|---|---|---|---|---|
| Challenge | No antibiotics | | | | No antibiotics (range) | | Lesion rate | Lesion score |
| (No. birds) | LPTas | RPTas | Trachea | Liver | Left + Right Airsac | Trachea | (>0.5) | (range) |
| E956 (20) | 2/20 | 1/20 | 8/20 | 0/20 | 0.00 (0-2.25)^{a} | 0.15 (0-1.88)^{a} | 13/20^{a} | 1.25 (0-4.5)^{a} |
| ΔoppD (20) | 1/20 | 0/20 | 9/20 | 0/20 | 0.00 (0-1.43)^{a} | 0.00 (0-2.80)^{a} | 6/20^{b} | 0.0 (0-14)^{b} |

Values with the same subscript symbols in the same column are not significantly different (P ≧ 0.05 by Mann-Whitney test or Fischer's exact 2-tailed test)

TABLE 20
Probability (P) values for statistical analysis of airsac
and tracheal re-isolation rates from table 19.

| | Airsac | Trachea |
|---|---|---|
| Strain | Chall only | Chall only |
| ΔoppD | 0.130 | 0.0004 |

Mann-Whitney test

TABLE 21
Probability (P) values for statistical analysis
of airsac lesion scores and rates from table 19.

| | Lesion Score* | Lesion Rate\hat{ } |
|---|---|---|
| Strain | Chall only | Chall only |
| ΔoppD | 0.011 | 0.052 |

*Mann-Whitney test,
\hat{ }Fischer's exact 2-tailed

### Weight Gain of Birds

The difference between the percentage weight gains in the pathogenicity experiment between E956 (143%), ΔdppD (143%) and non-vaccinated chickens (174%) was marginally significant whilst no significant difference was seen with ΔoppD (155%) to that of non-vaccinated birds (Table 14 and 15). In the efficacy experiment there was no statistically significant difference in the percentage weight gain of any bird group before challenge or at post mortem (Table 14 and 15).

### Re-Isolation Rates of Organisms on MacConkey Agar

There was no significant difference between the median numbers of organisms re-isolated from the airsac or trachea of control and ΔoppD vaccinated birds in the pathogenicity experiment (Table 16). There were higher numbers of organisms isolated from those birds vaccinated with E956 or ΔdppD than non-vaccinated birds (Table 16). The results for the efficacy experiment (Table 19) show no difference is between the E956 challenged and ΔoppD vaccinated birds. There was no significant difference in the rate of isolations made from the trachea versus airsacs in birds vaccinated with either ΔdppD or ΔoppD though a significant increase in isolation of organisms from the trachea compared to the airsacs was observed with birds vaccinated with E956 (P<0.05).

### Airsac Lesion Rates

Examination of the airsacs and organs at post mortem showed signs of colibacillosis in some birds with perihepatitis and pericarditis observed. In the pathogenicity experiment there was no statistically significant difference between birds vaccinated with either ΔdppD or ΔoppD in the rate and lesion score of the airsacs, there were less lesions observed between ΔoppD and E956 but no statistical difference between ΔdppD and E956 vaccinated birds (Table 16 and 18). In the protection experiment, comparison of lesion rate and scores of ΔoppD vaccinated and E956 challenged birds showed a statistically significant protective effect from prior vaccination with ΔoppD with a median lesion rate of 0.0 for vaccinates and 1.25 for the non-vaccinated and challenged group (Table 19 and 21).

1. A bacterium attenuated by a mutation in at least one ABC peptide transporter gene wherein the mutation renders the encoded ABC-peptide transporter protein non-functional and wherein the attenuated bacterium can persist in a subject.

2. The bacterium of claim 1 wherein the mutation is generated by insertion, deletion, substitution or any combination thereof.

3. The bacterium of claim 2 wherein the insertion mutation is made by any one or any combination of homologous recombination, transposon mutagenesis or sequence tag mutagenesis.

4. The bacterium of claim 1 wherein the ABC-peptide transporter gene is selected from the group consisting of CvaB, CylB, SpaB, NisT, EpiT, PedD, LcnC, McbEF, OppD and DppD, and their homologues.

5. The bacterium of claim 1 wherein the ABC-peptide transporter gene is OppD or DppD.

6. (canceled)

7. The bacterium of claim 1 wherein the bacterium is selected from the group comprising Avibacterium, Bacillus, Brucella, Bartonella, Bordetella, Burkholderia, Vibrio, Escherichia, Salmonella, Clostridium, Campylobacter, Chlamydia, Coxiella, Erysipelothrix, Francisella, Listeria, Actinobacillus, Haemophilus, Helicobacter, Aeromonas, Pseudomonas, Streptococcus, Shigella, Yersinia, Mycoplasma, Mycobacterium, Mannheimia, Ornithobacterium, Rickettsia, Ureaplasma and Pasteurella.

8. The bacterium of claim 7 wherein the bacterium is Avibacterium paragallinarum, Bordetella avium, Ornithobacterium rhinotracheale, Salmonella enteritidis, Pasteurella multocida, Mannheimia haemolytica, E. coli, Clostridium perfringens, Mycoplasma agalactiae, Mycoplasma alkalescens, Mycoplasma anatis, Mycoplasma anseris, Mycoplasma imitans, Mycoplasma arginini, Ureaplasma parvum, Mycoplasma arthritidis, Mycoplasma bovigenitalium, Mycoplasma bovirhinis, Mycoplasma bovis, Mycoplasma bovoculi, Mycoplasma californicum, Mycoplasma capricolum, Mycoplasma dispar, Mycoplasma fells, Mycoplasma fermentans, Mycoplasma genitalium, Mycoplasma hominis, Mycoplasma hyopneumoniae, Mycoplasma hyorhinis, Mycoplasma hyosynoviae, Mycoplasma iowae, Mycoplasma mycoides subsp mycoides large colony and small colony, Mycoplasma gallisepticum, Mycoplasma synoviae, Mycoplasma orale, Mycoplasma penetrans, Mycoplasma ovipneumoniae, Mycoplasma pullorum, Mycoplasma alligatorus, Mycoplasma pneumoniae, Mycoplasma, Mycoplasma maleagridis, Mycoplasma haemofelis, Mycoplasma haemominutum, Mycoplasma haematoparvum.

9. The bacterium of claim 1 wherein the bacterium is avian pathogenic E. coli strain E956, or Mycoplasma gallisepticum strain Ap3AS.

10. The bacterium of claim 1 wherein the attenuated bacterium expresses a heterologous antigen.

11. The bacterium of claim 10 wherein the heterologous antigen encodes by a nucleic acid from either the same or another pathogenic organism.

12. The bacterium of claim 11 wherein the nucleic acid encoding the heterologous antigen is isolated from genera selected from the group comprising, Avibacterium, Bacillus, Brucella, Bartonella, Bordetella, Burkholderia, Vibrio, Escherichia, Salmonella, Clostridium, Campylobacter, Chlamydia, Coxiella, Erysipelothrix, Francisella, Listeria, Actinobacillus, Haemophilus, Helicobacter, Aeromonas, Pseudomonas, Streptococcus, Shigella, Yersinia, Mycoplasma, Mycobacterium, Mannheimia, Ornithobacterium, Rickettsia, Ureaplasma and Pasteurella or any combination thereof.

13. The bacterium of claim 12 wherein the nucleic acid encoding the heterologous antigen is derived from Avibacterium paragallinarum, Bordetella avium, Ornithobacterium rhinotracheale, Salmonella enteritidis, Pasteurella multocida, Mannheimia haemolytica, E. coli, Clostridium perfringens, Mycoplasma agalactiae, Mycoplasma alkalescens, Mycoplasma anatis, Mycoplasma anseris, Mycoplasma imitans, Mycoplasma arginini, Ureaplasma parvum, Mycoplasma arthritidis, Mycoplasma bovigenitalium, Mycoplasma bovirhinis, Mycoplasma bovis, Mycoplasma bovoculi, Mycoplasma califormicum, Mycoplasma capricolum, Mycoplasma dispar, Mycoplasma fells, Mycoplasma fermentans, Mycoplasma genitalium, Mycoplasma hominis, Mycoplasma hyopneumoniae, Mycoplasma hyorhinis, Mycoplasma hyosynoviae, Mycoplasma iowae, Mycoplasma mycoides subsp mycoides large colony and small colony, Mycoplasma gallisepticum, Mycoplasma synoviae, Mycoplasma orale, Mycoplasma penetrans, Mycoplasma ovipneumoniae, Mycoplasma pullorum, Mycoplasma alligatorus, Mycoplasma pneumoniae, Mycoplasma, Mycoplasma maleagridis, Mycoplasma haemofelis, Mycoplasma haemominutum, Mycoplasma haematoparvum or any combination thereof.

14. The bacterium of claim 11 wherein the nucleic acid encoding the heterologous antigen is isolated from viruses selected from the group comprising Newcastle Disease virus, Infectious Bronchitis virus, Avian Pneumovirus, Fowlpox, Infectious Bursal Disease, Infectious Laryngotracheitis Virus, Avian Influenza, Duck Virus Hepatitis, Duck Plague, Chicken Infectious Anaemia, Marek's Disease Virus or any combination thereof.

15. The bacterium of claim 1 wherein the subject is a vertebrate animal selected from the group consisting of humans, bovines, canines, felines, caprines, ovines, porcines, camelids, equines and avians.

16. (canceled)

17. The bacterium of claim 15 wherein the bovine subject is a cow, ox, bison or buffalo, the canine subject is a dog, the feline subject is a cat, the caprine subject is a goat, the ovine subject is a sheep, the porcine subject is a pig, the camelid subject is a camel, dromedary, llama, alpaca, vicuña or guanaco, the equine subject is a horse, donkey, zebra or mule.

18. (canceled)

19. The bacterium of claim 15 wherein the subject is an avian subject selected from chickens (including bantams), turkeys, ducks, geese, pheasants, quails, partridges, pigeons, guinea-fowls, ostriches, emus or pea-fowl.

20. (canceled)

21. A method of prevention or amelioration of a disease in a subject, the method comprising administering a therapeutically effective dose of a vaccine composition or immunogenic composition to the subject wherein the vaccine composition or immunogenic composition comprises at least one attenuated bacteria of claim 1.

22. A method of prophylaxis of a disease, the method comprising administering a therapeutically effective dose of a vaccine composition or immunogenic composition to a subject in need of prophylaxis wherein said vaccine composition or immunogenic composition comprises at least one attenuated bacteria of claim 1.

23. An immunogenic composition comprising at least one attenuated bacteria of claim 1.

24. A vaccine composition comprising an immunogenically effective amount of at least one attenuated bacteria of claim 1 and a pharmacologically acceptable carrier.

25-38. (canceled)


SEQUENCE LISTING (91 sequences)

SEQ ID NO: 1 - DNA, 40 bp, Artificial
  synthetic signature tag oligonucleotide Tag 01 (Ptag5)
ccacagcgctttctcccgcgcccaatcacccaccctcaat

SEQ ID NO: 2 - DNA, 39 bp, Artificial Sequence
  synthetic signature tag oligonucleotide Tag 02 (Ptag7)
agctacaagcgaactacctcccgctatcacccgctcgca

SEQ ID NO: 3 - DNA, 40 bp, Artificial Sequence
  synthetic signature tag oligonucleotide Tag 03 (Ptag9)
acccataccgaaacaactctcccgcaagcgcccaacccct

SEQ ID NO: 4 - DNA, 40 bp, Artificial Sequence
  synthetic signature tag oligonucleotide Tag 04 (Ptag10), Tag 08 (1R1-5)
atcgaggtatcggttgctagggggtacgctagggggagtt

SEQ ID NO: 5 - DNA, 40 bp, Artificial Sequence
  synthetic signature tag oligonucleotide Tag 05 (Ptag12), Tag 17 (R3-9)
ctgggggtggagaggtcggtgattaggagtctcggtgtta

SEQ ID NO: 6 - DNA, 40 bp, Artificial Sequence
  synthetic signature tag oligonucleotide Tag 06 (Ptag17)
agagcttgaaatattgggcgcgagtgttcagggttggggt

SEQ ID NO: 7 - DNA, 40 bp, Artificial Sequence
  synthetic signature tag oligonucleotide Tag 07 (Ptag20)
agatgaggggctggagctagagatgctgatttagcggagg

SEQ ID NO: 8 - DNA, 40 bp, Artificial Sequence
  synthetic signature tag oligonucleotide Tag 09 (1R1-10)
atctatccaccgatactcctctcaatctagcgcgatacct

SEQ ID NO: 9 - DNA, 40 bp, Artificial Sequence
  synthetic signature tag oligonucleotide Tag 10 (1R1-20)
gtcgtgcgtgggagagggctatggatggttgatgaggggt

SEQ ID NO: 10 - DNA, 40 bp, Artificial Sequence
  synthetic signature tag oligonucleotide Tag 11 (1R1-29)
ctatatctacatctctcacccgagcgctccccaaaccgcc

SEQ ID NO: 11 - DNA, 40 bp, Artificial Sequence
  synthetic signature tag oligonucleotide Tag 12 (R2-3)
ctccacctctatctctctatccccacatagacccacctcc

SEQ ID NO: 12 - DNA, 41 bp, Artificial Sequence
  synthetic signature tag oligonucleotide Tag 13 (R2-5)
cctatcacgcttcctccttcaacctaactcccactagagct

SEQ ID NO: 13 - DNA, 40 bp, Artificial Sequence
  synthetic signature tag oligonucleotide Tag 14 (R2-9)
atctccctatctccctcgagctcttgccctctaacaagct

SEQ ID NO: 14 - DNA, 41 bp, Artificial sequence
  synthetic signature tag oligonucleotide Tag 15 (R2-18)
gtaattcgttcggtatttattggtctggcggtaggtagtag

SEQ ID NO: 15 - DNA, 41 bp, Artificial Sequence
  synthetic signature tag oligonucleotide Tag 16 (R2-22)
ctccctctactcccccgatacacacccctacccaccaatcc

SEQ ID NO: 16 - DNA, 40 bp, Artificial sequence
  synthetic signature tag oligonucleotide Tag 18 (R3-17)
aaacccctgccacgtcagcgatctccctcacactagctct

SEQ ID NO: 17 - DNA, 40 bp, Artificial Sequence
  synthetic signature tag oligonucleotide Tag 19 (R3-23)
ctcccattcccactcacccgatctttgccactccaaaccc

SEQ ID NO: 18 - DNA, 40 bp, Artificial Sequence
  synthetic signature tag oligonucleotide Tag 20 (R3-24)
actacgacatatcctacactacaacgctacatcccgcgct

SEQ ID NO: 19 - DNA, 39 bp, Artificial Sequence
  synthetic signature tag oligonucleotide Tag 21 (R3-33)
ggagatgttcaagttagggaggtggctgtaggtagagag

SEQ ID NO: 20 - DNA, 40 bp, Artificial Sequence
  synthetic signature tag oligonucleotide Tag 22 (R3-35)
akcggtatgtcggtagagtggttggggtaggaggagagcg

SEQ ID NO: 21 - DNA, 41 bp, Artificial Sequence
  synthetic signature tag oligonucleotide Tag 23 (R3-47)
agagcgggatagtgtagttatcgttaggtcttgtggtttta

SEQ ID NO: 22 - DNA, 40 bp, Artificial Sequence
  synthetic signature tag oligonucleotide Tag 24 (R3-50)
agagatttatcgatagcgaggtcgtgctggcgagtgggtg

SEQ ID NO: 23 - DNA, 27 bp, Artificial Sequence
  synthetic signature tag oligonucleotide Tag 25 (R3-54)
gtcgggagggagaagatcagtgtgttg

SEQ ID NO: 24 - DNA, 40 bp, Artificial Sequence
  synthetic signature tag oligonucleotide Tag 26 (R3-57)
agagaagtatcgggatgtcgctcgcgcttgtgtgggggaa

SEQ ID NO: 25 - DNA, 40 bp, Artificial Sequence
  synthetic signature tag oligonucleotide Tag 27 (R3-66)
gtatgggtagggggagagggagctagtgatttacagggat

SEQ ID NO: 26 - DNA, 40 bp, Artificial Sequence
  synthetic signature tag oligonucleotide Tag 28 (R23)
ggcggggtgtagagagattgggagggatgtacagcgggtt

SEQ ID NO: 27 - DNA, 40 bp, Artificial Sequence
  synthetic signature tag oligonucleotide Tag 29 (R26)
cgggagatagaagttttgagagggagagttagattgagtg

SEQ ID NO: 28 - DNA, 40 bp, Artificial Sequence
  synthetic signature tag oligonucleotide Tag 30 (R32)
ggtgtgggcgcttagttgggcaagcttgcgtggtgggtct

SEQ ID NO: 29 - DNA, 41 bp, Artificial Sequence
  synthetic signature tag oligonucleotide Tag 31 (R43)
aggtagatatctatagaggtagttaggctggggatcgagga

SEQ ID NO: 30 - DNA, 40 bp, Artificial Sequence
  synthetic signature tag oligonucleotide Tag 32 (R46)
ctctatccatctcgtaacccatatcgatagatcgcaatac

SEQ ID NO: 31 - DNA, 39 bp, Artificial Sequence
  synthetic signature tag oligonucleotide Tag 33 (R52)
cgatacagacccgaaatctctcaaccccgcacgcaaact

SEQ ID NO: 32 - DNA, 40 bp, Artificial Sequence
  synthetic signature tag oligonucleotide Tag 34 (R56)
atctagcaccaacactatccatagcccactatatatattc

SEQ ID NO: 33 - DNA, 40 bp, Artificial Sequence
  synthetic signature tag oligonucleotide Tag 35 (R62)
gggggtagagcgtgagcgggatgtgtcggggggtagggag

SEQ ID NO: 34 - DNA, 40 bp, Artificial Sequence
  synthetic signature tag oligonucleotide Tag 36 (R75)
aaagctctggtgtctgggcgctagggagggatcgaggagg

SEQ ID NO: 35 - DNA, 40 bp, Artificial Sequence
  synthetic signature tag oligonucleotide Tag 37 (R77)
cccacactctccgacccctcagatcccccccgccaaccct

SEQ ID NO: 36 - DNA, 40 bp, Artificial Sequence
  synthetic signature tag oligonucleotide Tag 38 (R79)
agggcgagatgtaggtgtatggagctggggatggagataa

SEQ ID NO: 37 - DNA, 41 bp, Artificial Sequence
  synthetic signature tag oligonucleotide Tag 39 (R80)
ccaactcctccgtacacccccgctagcaccccaatccctcg

SEQ ID NO: 38 - DNA, 18 bp, Artificial Sequence
  synthetic oligonucleotide primer P2 for signature tag region PCR
atcctacaacctcaagct

SEQ ID NO: 39 - DNA, 18 bp, Artificial Sequence
  synthetic oligonucleotide primer P4 for signature tag region PCR
atcccattctaaccaagc

SEQ ID NO: 40 - DNA, 26 bp, Artificial Sequence
  synthetic oligonucleotide primer IGstmGenmeF3 for DNA sequencing ST mutant PCR
ggactgttatatggcctttttggatc

SEQ ID NO: 41 - DNA, 20 bp, Artificial Sequence
  synthetic oligonucleotide MG-F for Mg 16S gene PCR
gttgcaaatccgtaaggtgg

SEQ ID NO: 42 - DNA, 20 bp, Artificial Sequence
  synthetic oligonucleotide MG-R for Mg 16S gene PCR
ttagcaacacggttttagat

SEQ ID NO: 43 - DNA, 26 bp, Artificial Sequence
  synthetic oligonucleotide EOGentSpeIRev for Gentamycin gene PCR
actagtatcagccaatcgcttaattg

SEQ ID NO: 44 - DNA, 26 bp, Artificial Sequence
  synthetic oligonucleotide EOGentSpeIFor for Gentamycin gene PCR
actagtctgagtttatggaagaagtt

SEQ ID NO: 45 - DNA, 20 bp, Artificial Sequence
  synthetic oligonucleotide M13-For for DNA sequencing
gttgtaaaacgacggccagt

SEQ ID NO: 46 - DNA, 18 bp, Artificial Sequence
  synthetic oligonucleotide M13-Rev for DNA sequencing
caggaaacagctatgacc

SEQ ID NO: 47 - DNA, 20 bp, Artificial Sequence
  synthetic oligonucleotide STM02-B-Rev for ST mutant PCR
ttcaattcgaaaaatgagtt

SEQ ID NO: 48 - DNA, 18 bp, Artificial Sequence
  synthetic oligonucleotide STM03-B-Rev for ST mutant PCR
acagcttgacgttttcca

SEQ ID NO: 49 - DNA, 20 bp, Artificial Sequence
  synthetic oligonucleotide STM04-B-1 for ST mutant PCR
cggggacacagtaaggctaa

SEQ ID NO: 50 - DNA, 18 bp, Artificial Sequence
  synthetic oligonucleotide STM04-C-Rev for ST mutant PCR
cgcattgattcctaccac

SEQ ID NO: 51 - DNA, 20 bp, Artificial Sequence
  synthetic oligonucleotide STM06-A-Rev for ST mutant PCR
aacatggattagacgtttgc

SEQ ID NO: 52 - DNA, 21 bp, Artificial Sequence
  synthetic oligonucleotide STM09-B-Rev for ST mutant PCR
tgcctttcttaatagtgctca

SEQ ID NO: 53 - DNA, 18 bp, Artificial Sequence
  synthetic oligonucleotide STM10-Rev for ST mutant PCR
ttgtgcccattcgttagt

SEQ ID NO: 54 - DNA, 22 bp, Artificial Sequence
  synthetic oligonucleotide STM13-JA-rev for ST mutant 13 sequencing
ttaatacgcctgggtaaggtcg

SEQ ID NO: 55 - DNA, 23 bp, Artificial Sequence
  synthetic oligonucleotide STM13-JB-for for ST mutant 13 sequencing
tatctatcacgtaattggaatgc

SEQ ID NO: 56 - DNA, 21 bp, Artificial Sequence
  synthetic oligonucleotide STM13-KE for ST mutant 13 sequencing
tcgctataacatttgatttag

SEQ ID NO: 57 - DNA, 19 bp, Artificial Sequence
  synthetic oligonucleotide STM13-KE-C′ for ST mutant 13 sequencing
cattttgatgtttgcgtca

SEQ ID NO: 58 - DNA, 17 bp, Artificial Sequence
  synthetic oligonucleotide STM13-KE-C′-1 for ST mutant 13 sequencing
cttcaagttctctgtgg

SEQ ID NO: 59 - DNA, 17 bp, Artificial Sequence
  synthetic oligonucleotide STM13-KE-C′-1-Rev for Wild-type Ap3AS PCR
ccacagagaacttgaag

SEQ ID NO: 60 - DNA, 25 bp, Artificial Sequence
  synthetic oligonucleotide STM13-KF for ST mutant 13 sequencing
ccgtttttcttttattcgtattatc

SEQ ID NO: 61 - DNA, 16 bp, Artificial Sequence
  synthetic oligonucleotide STM13-KF-C′ for ST mutant 13 sequencing and Wild-type Ap3AS PCR
tataaacctggtacgg

SEQ ID NO: 62 - DNA, 19 bp, Artificial Sequence
  synthetic oligonucleotide STM13-KF-C′-1 for ST mutant 13 sequencing
cacactgaaagttatatta

SEQ ID NO: 63 - DNA, 18 bp, Artificial Sequence
  synthetic oligonucleotide STM13-KF-C′-2 for ST mutant 13 sequencing
ctcactatataaaatagc

SEQ ID NO: 64 - DNA, 21 bp, Artificial Sequence
  synthetic oligonucleotide STM13-KG for ST mutant 13 sequencing
cagaagataagtctttaacag

SEQ ID NO: 65 - DNA, 28 bp, Artificial Sequence
  synthetic oligonucleotide JX-STM17 for ST mutant PCR
ctagaagaagaagaagaggtaacgaagc

SEQ ID NO: 66 - DNA, 20 bp, Artificial Sequence
  synthetic oligonucleotide STM18-Rev for ST mutant PCR
agcaaaatttccacccaaga

SEQ ID NO: 67 - DNA, 20 bp, Artificial Sequence
  synthetic oligonucleotide STM20-Rev for ST mutant PCR
gagacatcaatcgcggtttt

SEQ ID NO: 68 - DNA, 23 bp, Artificial Sequence
  synthetic oligonucleotide JZ-STM22 for ST mutant PCR
ctcaattgaagaattatatgatg

SEQ ID NO: 69 - DNA, 18 bp, Artificial Sequence
  synthetic oligonucleotide STM23-B-Rev for ST mutant PCR
ttaaccccattcttgcag

SEQ ID NO: 70 - DNA, 20 bp, Artificial Sequence
  synthetic oligonucleotide STM26-Rev for ST mutant PCR and Wild-type Ap3AS PCR
catcccacctgaaaactcgt

SEQ ID NO: 71 - DNA, 20 bp, Artificial Sequence
  synthetic oligonucleotide STM26-genome for Wild-type Ap3AS PCR
gatcggtgcacagattaaag

SEQ ID NO: 72 - DNA, 19 bp, Artificial Sequence
  synthetic oligonucleotide KB-STM33 for ST mutant PCR
actacacggtagttagtac

SEQ ID NO: 73 - DNA, 20 bp, Artificial Sequence
  synthetic oligonucleotide primer TXkanFor
tagactgggcggttttatgg

SEQ ID NO: 74 - DNA, 20 bp, Artificial Sequence
  synthetic oligonucleotide primer TYkanRev
cgaagcccaacctttcatag

SEQ ID NO: 75 - DNA, 20 bp, Artificial Sequence
  synthetic oligonucleotide primer TZ5′dppD
cgtagaccgcatcagctaca

SEQ ID NO: 76 - DNA, 20 bp, Artificial Sequence
  synthetic oligonucleotide primer UA3′dppD
gtgggtaatggcatttggac

SEQ ID NO: 77 - DNA, 20 bp, Artificial Sequence
  synthetic oligonucleotide WAKanFshort
cgttggctacccgtgatatt

SEQ ID NO: 78 - DNA, 60 bp, Artificial Sequence
  synthetic oligonucleotide WE_dppD5′_kanF
ccgtcagcgagcgattgacctgctgaatcaggtcggtatttagactgggcggttttatgg

SEQ ID NO: 79 - DNA, 60 bp, Artificial Sequence
  synthetic oligonucleotide WF_dppD3′_kanR
gcgggttaagcaggcagccgttcgggcggtcgtacttgcccgaagcccaacctttcatag

SEQ ID NO: 80 - DNA, 20 bp, Artificial Sequence
  synthetic oligonucleotide WSoppDFor
gcactgctgaacgtgaaaga

SEQ ID NO: 81 - DNA, 20 bp, Artificial Sequence
  synthetic oligonucleotide WToppDRev
ctccaccggtttaaagcaag

SEQ ID NO: 82 - DNA, 40 bp, Artificial Sequence
  synthetic oligonucleotide WU1oppDRev+Kan
ccataaaaccgcccagtctatttttaccgcatcgagcatc

SEQ ID NO: 83 - DNA, 40 bp, Artificial Sequence
  synthetic oligonucleotide WV2oppDFor+Kan
ctatgaaaggttgggcttcgtcgacctaagctgctgattg

SEQ ID NO: 84 - DNA, 20 bp, Artificial Sequence
  synthetic oligonucleotide Sidfwdseq
ctttccaccctgcacctaag

SEQ ID NO: 85 - DNA, 20 bp, Artificial Sequence
  synthetic oligonucleotide bwd siderophore
ctcacgggtgaaaatatttt

SEQ ID NO: 86 - DNA, 20 bp, Artificial Sequence
  synthetic oligonucleotide 16Sfwd
gctgacgagtggcggacggg

SEQ ID NO: 87 - DNA, 20 bp, Artificial Sequence
  synthetic oligonucleotide 16Srev
taggagtctggaccgtgtct

SEQ ID NO: 88 - DNA, 40 bp, Artificial Sequence
  synthetic oligonucleotide DNA signature tag
nknknknknknknknknknknknknknknknknknknknk

SEQ ID NO: 89 - DNA, 82 bp, Artificial Sequence
  synthetic signature-tagged (ST) mutant 26-2 by direct sequencing
cacaataatacggacttgatctaaagatcaatatctaaaacaaaaacaagaattagaaaaacaccaactctaatagtgctga

SEQ ID NO: 90 - DNA, 90 bp, Artificial Sequence
  synthetic signature-tagged (ST) mutant 15-1 by direct sequencing
cacaataatacggacttgatctantattgngatncttnatnatgatntgcaataaattacacagattctntacttcatttatatacatcc

SEQ ID NO: 91 - DNA, 80 bp, Artificial Sequence
  synthetic signature-tagged (ST) mutant 25-1 by direct sequencing
cacaataatacggacttgatctggtttgttaacctanancaanatntnctancnggnnacacnntnatgcgctatgtatc